(19) United States
(12) Patent Application Publication
(10) Pub. No.: US 2025/0345197 A1
(43) Pub. Date: Nov. 13, 2025

(54) IMPLANT DELIVERY AND DELIVERY SYSTEM RETRIEVAL

(71) Applicant: EDWARDS LIFESCIENCES CORPORATION, Irvine, CA (US)

(72) Inventors: Roei Mayo, Irvine, CA (US); Ben Shitrit, Eilat (IL); Daniel James Murray, Orange, CA (US)

(21) Appl. No.: 19/273,099

(22) Filed: Jul. 17, 2025

Related U.S. Application Data
(63) Continuation of application No. PCT/US2024/012057, filed on Jan. 18, 2024.
(60) Provisional application No. 63/481,108, filed on Jan. 23, 2023.

(51) Int. Cl. A61F 2/966

(52) U.S. Cl. CPC A61F 2/966

(57) ABSTRACT

An implant delivery system includes an elongate sheath having an inner diameter at a distal end thereof, a nose cone shaft dimensioned for advancement within the elongate sheath, and a collapsible nose cone associated with a distal end of the nose cone shaft and positionable at least partially beyond the distal end of the elongate sheath, the collapsible nose cone being transitionable between an expanded state having a first maximum diameter and a compressed state having a second maximum diameter that is less than the inner diameter of the elongate sheath.

### RELATED APPLICATION(S)

This application is a continuation of International Patent Application No. PCT/US24/12057, filed Jan. 18, 2024, which claims the benefit of U.S. Provisional Patent Application No. 63/481,108, filed Jan. 23, 2023, the complete disclosures of which are hereby incorporated by reference in their entireties.

### BACKGROUND

The present disclosure generally relates to the field of delivery systems for medical implant devices. Implant devices can be advanced to target anatomy using percutaneous and/or minimally-invasive access. For example, transcatheter procedures can be implemented to transport an implant device through the vasculature of a patient using an elongated tubular delivery system. The particular configuration of implant delivery systems and/or components thereof can affect the efficiency, risks, and/or efficacy associated with device implantation procedures.

### SUMMARY

Described herein are devices, methods, and systems that facilitate the delivery and/or deployment of certain implant devices, including implant devices that have at least one low-profile/narrow dimension, such as stents having a non-circular biased cross-sectional shape, which may be utilized for blood vessel compliance enhancement or other purposes. Furthermore, the present disclosure provides devices, methods, and systems that facilitate retrieval of distal nose cone components/features of delivery systems after implant deployment. Devices associated with the various examples of the present disclosure can include delivery system shafts/lumens that have a non-circular axial cross-sectional shape to accommodate stents or other implant devices that have non-circular natural cross-sectional shapes.

Furthermore, various examples of the present disclosure provide distal nose cones for delivery systems, wherein such nose cones have features that facilitate introduction and/or advancement through/along transcatheter/percutaneous access paths. Nose cone examples presented herein further include nose cones having, or configured to assume, a low-profile diameter that is less than a diameter of a delivery shaft/lumen of the delivery system associated with the nose cone. Such reduced nose cone profile/diameter can facilitate retrieval/removal of the nose cone through a deployed implant without the nose cone becoming caught or otherwise interfered with by the implant (e.g., stent) when the nose cone is withdrawn therethrough. Nose cone profile reduction means/mechanisms in accordance with aspects of the present disclosure can comprise nose cone and/or sheath cover features, deflatable nose cones, and the like. Some nose cones disclosed herein include proximal taper features to guide the nose cone back into/through a lumen of a deployed implant device with reduced risk of catching on the implant.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates example cardiac and vascular anatomy of a patient having a healthy, compliant aorta.

FIGS. 2A and 2B show side and axial cross-sectional views, respectively, of the healthy aorta of FIG. 1 experiencing compliant expansion.

FIG. 3 shows an example stiff aorta.

FIGS. 4A and 4B show side and axial cross-sectional views, respectively, of the stiff aorta of FIG. 3 experiencing compromised expansion.

FIGS. 5-1 and 5-2 show a blood vessel in circular and non-circular shapes, respectively.

FIG. 6 shows a perspective views of a non-circular stent in accordance with one or more examples.

FIGS. 7A, 7B, and 7C show side and axial views, respectively, of a delivery system having a non-circular stent disposed therein in accordance with one or more examples.

FIGS. 8A and 8B show side and axial views, respectively, of a delivery system having a stent disposed therein in accordance with one or more examples.

FIG. 9 shows a delivery system deploying a stent implant in accordance with one or more examples.

FIGS. 10A, 10B, and 10C show side and perspective views, respectively, of a delivery system having a nose cone with a diameter that is greater than a minor-axis diameter of a non-circular stent in accordance with one or more examples.

FIGS. 11A, 11B, and 11C show axial, major-axis side, and minor-axis side views, respectively, of a non-circular delivery system including a nose cone having a non-circular base in accordance with one or more examples.

FIGS. 12A, 12B, and 12C show axial, major-axis side, and minor-axis side views, respectively, of a non-circular, transitional nose cone having a non-circular base in accordance with one or more examples.

FIG. 13 shows a side cutaway view of a delivery system including an inflatable nose cone in accordance with one or more examples,

FIGS. 14-1, 14-2, 14-3, and 14-4 illustrate a flow diagram for a process for deploying an implant device using a delivery system with an inflatable nose cone in accordance with one or more examples.

FIGS. 15-1, 15-2, 15-3, and 15-4 provide images of the delivery system and certain anatomy corresponding to operations of the process of FIGS. 14-1, 14-2, 14-3, and 14-4 according to one or more examples.

FIG. 16 shows a nose cone having a proximal step in accordance with one or more examples.

FIG. 17 shows a nose cone having a tapered proximal end in accordance with one or more examples.

FIG. 18 shows a nose cone having transitional tapering in accordance with one or more examples.

FIG. 19 shows a delivery system including a narrow nose cone and a flexible cover in accordance with one or more examples.

FIGS. 20-1, 20-2, 20-3, and 20-4 illustrate a flow diagram for a process for deploying an implant device using a delivery system with a flexible nose cone cover in accordance with one or more examples.

FIGS. 21-1, 21-2, 21-3, and 21-4 provide images of the delivery system and certain anatomy corresponding to operations of the process of FIGS. 20-1, 20-2, 20-3, and 20-4 according to one or more examples.

FIG. 22 shows a delivery system including a nose cone comprising a proximal flexible cover in accordance with one or more examples.

FIG. 23 shows the delivery system of FIG. 22 deploying a stent implant in accordance with one or more examples,

FIGS. 24A and 24B show perspective views of a collapsible nose cone including a frame with struts in accordance with one or more examples.

FIG. 25 shows the nose cone frame of FIGS. 24A and 24B in a collapsed state in according with one or more examples.

FIGS. 26A and 26B show perspective views of a collapsible nose cone including a frame and cover in accordance with one or more examples.

FIGS. 27A and 27B show perspective views of a collapsible nose cone including a coil frame in accordance with one or more examples.

FIG. 28 shows the nose cone frame of FIGS. 27A and 27B in a collapsed state in according with one or more examples.

FIGS. 29A and 29B show perspective views of a collapsible nose cone including a frame and cover in accordance with one or more examples.

FIG. 30 shows a delivery system comprising a collapsible nose cone and a handle-based actuator configured to collapse and/or expand the nose cone in accordance with one or more examples.

FIGS. 31-1, 31-2, 31-3, and 31-4 illustrate a flow diagram for a process for deploying an implant device using a delivery system with a collapsible nose cone in accordance with one or more examples.

FIGS. 32-1, 32-2, 32-3, and 32-4 provide images of the delivery system and certain anatomy corresponding to operations of the process of FIGS. 31-1, 31-2, 31-3, and 31-4 according to one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, compliance-enhancing stent implant devices implanted/implantable in the aorta. However, although certain principles disclosed herein may be particularly applicable to the anatomy of the aorta, it should be understood that delivery systems, low-profile nose cones, and compliance-enhancement implant devices in accordance with the present disclosure may be implemented/implanted in, or configured for implementation/implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

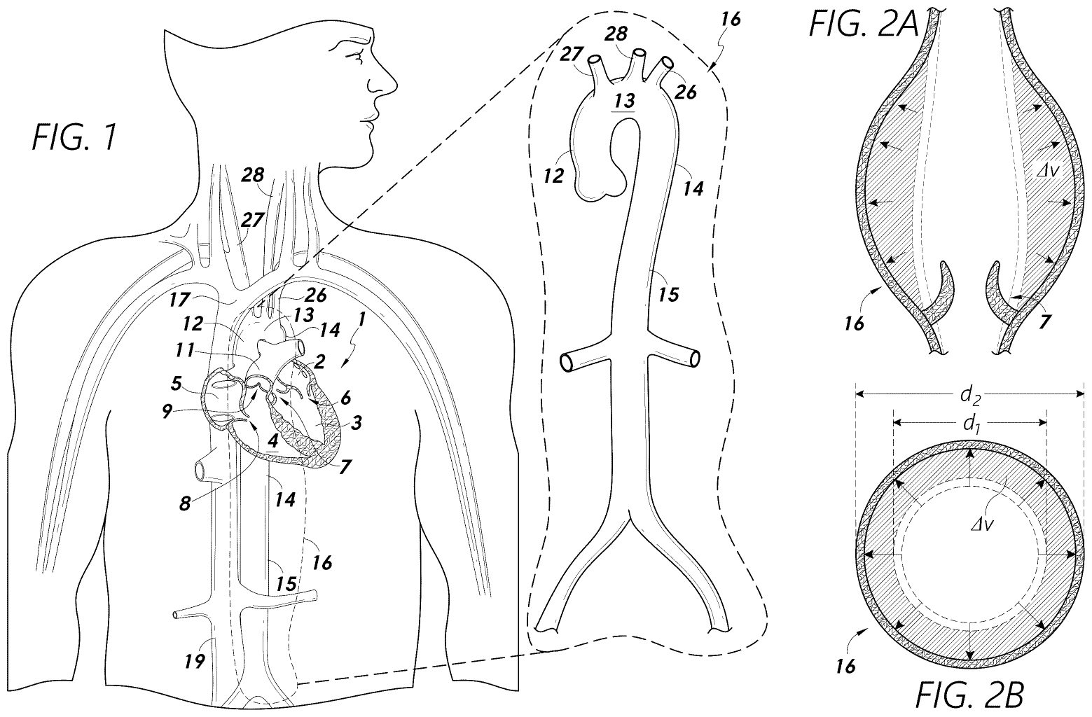

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3. A wall of muscle 17, referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior and superior venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery; together, these two arteries supply the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial (or venous) blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

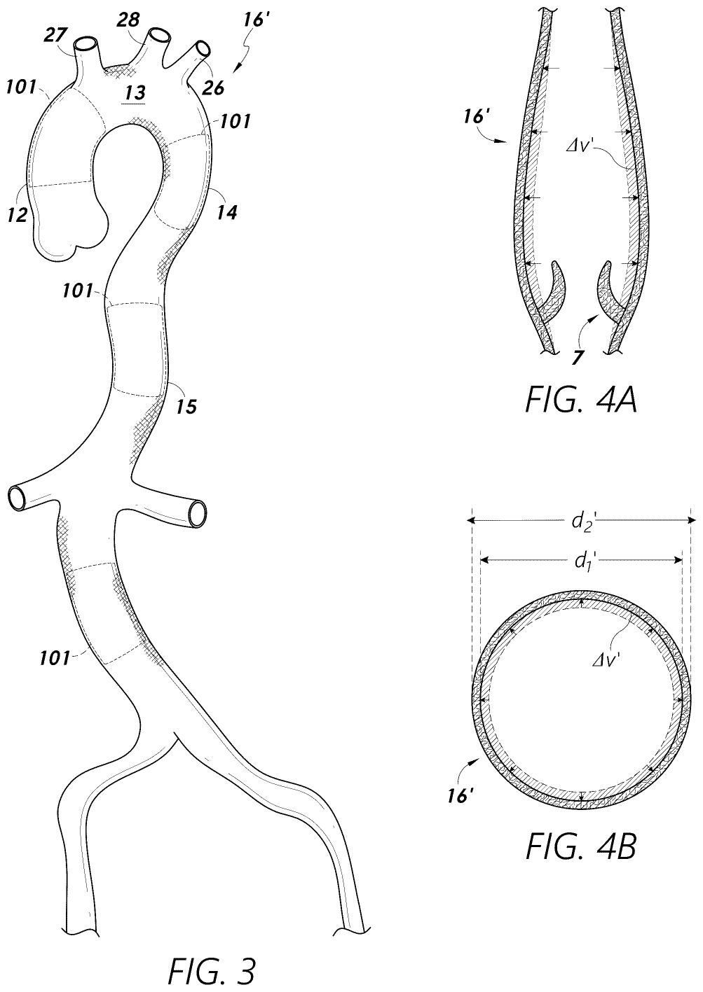

FIGS. 2A and 2B show side and axial cross-sectional views, respectively, of the healthy aorta 16 of FIG. 1 experiencing compliant expansion and contraction over a cardiac cycle. FIG. 3 shows an example stiff aorta 16′, whereas FIGS. 4A and 4B show side and axial cross-sectional views, respectively, of the stiff aorta 16′ of FIG. 3 experiencing compromised expansion and contraction over a cardiac cycle.

The systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Av will generally occur in an artery when the pressure in the artery is increased from diastole to systole. With respect to the aorta, as blood is pumped into the aorta 16 through the aortic valve 7, the pressure in the aorta increases and the diameter of at least a portion of the aorta expands. A first portion of the blood entering the aorta 16 during systole may pass through the aorta during the systolic phase, while a second portion (e.g., approximately half of the total blood volume) may be stored in the expanded volume Av caused by compliant stretching of the blood vessel 16, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Healthy arterial compliance may cause retraction/recoil of the blood vessel wall inward during diastole, thereby creating pressure in the blood vessel to cause blood to continue to be pushed through the artery 16 when the valve 7 is closed. For example, during systole, approximately 50% of the blood that enters the artery 16 through the valve 7 may be passed through the artery, whereas the remaining 50% may be stored in the artery, as enabled by expansion of the vessel wall. Some or all of the stored portion of blood in the artery 16 may be pushed through the artery by the contracting vessel wall during diastole. For patients experiencing arterial stiffness that causes lack of compliance, their arteries may not operate effectively in accordance with the expansion/contraction functionality shown in FIGS. 2A and 2B.

As shown in FIG. 3, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect, such as the thickening of the left ventricle muscle and/or diastolic heart failure, Stiffness in the aorta and/or other blood vessel(s) can occur due to an increase in collagen content and/or a corresponding decrease in elastin. While stiff/non-compliant blood vessels can generally suffer from a lack of elasticity in the walls thereof, as shown as causing compromised/reduced stretching and volume change Δν′, such vessels can maintain some amount of flexibility/bendability, such that reshaping of the blood vessels can occur without necessarily requiring the stretching of the walls of the blood vessel.

Examples of the present disclosure provide delivery systems that can be used for deploying compliance-enhancing stent implant devices, which may be implanted in one or more locations in a compromised aorta and/or other vessel(s). For example, FIG. 3 shows example positions of implant devices 101 (e.g., non-circular stent devices) implanted in various areas of an aorta 16′, wherein example delivery systems of the present disclosure can be used to deliver the relevant implant d.

### Compliance-Enhancing Stent Implants

The present disclosure relates to delivery systems and methods for delivering various prosthetic implant devices in anatomy, such as vasculature, of a patient. As an example, implant devices that can be delivered using systems, devices, and methods disclosed herein can include stent or other implant devices configured to add-back and/or increase compliance in the aorta or other arterial (or venous) blood vessel(s) to provide improved perfusion of the heart muscle and/or other organ(s) of the body. For example, example implant devices that can be delivered using delivery systems of the present disclosure can include stents that, when implanted, are configured to decrease the cross-sectional area/volume of the blood vessel segment in which the stent is implanted during low-pressure conditions, such as diastole, which serves to force blood through the blood vessel segment by pushing the blood through the vessel as the vessel volume reduces in connection with stent contraction induced by cyclical drops in blood pressure.

The non-circular (e.g., oval- and/or peanut-shaped) stents that can be implanted with delivery systems of the present disclosure can advantageously be configured to generate a differential cross-sectional area or volume of the target blood vessel(s) (e.g., aorta) between high- and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel.

Using non-circular stents to produce complaint blood vessel volume change by manipulating/reshaping the native blood vessel walls can increase compliance in a target blood vessel without requiring blood vessel grafting or resection. Therefore, compared to blood flow solutions involving blood vessel grafting/resection, transcatheter delivery system examples of the present disclosure can provide solutions that avoids the risks that may be associated with cutting of the vessel and/or devices grafted in/to such vessels, which may present risk of rupture and blood leakage outside of the circulatory system.

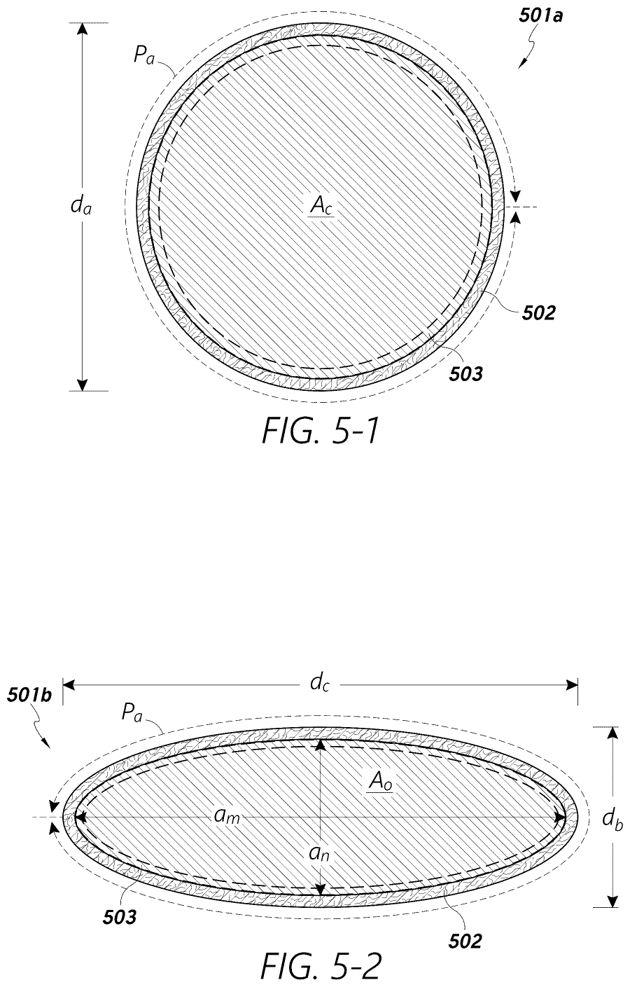

With respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area and volume of the blood vessel. FIG. 5-1 shows an example blood vessel 501 (identified as blood vessel 501a in FIG. 5-1) having a generally circular cross-sectional shape formed by the blood vessel wall 502, such that the area A_{c} thereof is maximized for the given perimeter/wall-length Pₐ. In the circular configuration, the diameter dₐ is substantially constant at every angle about the axis of the vessel. The circular shape of the vessel 501a may be set or permitted by the shape of a stent 503 implanted within the vessel.

Diverging from a circular cross-sectional shape can produce a cross-sectional area/volume for a blood vessel that is less than the maximum area A_{c} shown in FIG. 5-1. For example, FIG. 5-2 shows the blood vessel 501 (identified as vessel 501b in FIG. 5-2) having a shape that resembles an oval/ellipse, which produces the cross-sectional area Aₒ that is less than the area A_{c} with the same blood vessel wall/perimeter length Pₐ. The oval shape of the vessel 501b may have a major axis aₘ having a dimension d_{c} that is greater than a dimension d_{b} of the minor axis aₙ thereof. The oval shape of the vessel 501b may be set/forced by the stent 503, which may have a biased oval shape.

With further reference to FIGS. 5-1 and 5-2, due to the area Aₒ of the oval vessel of FIG. 5-1 being less than the area A_{c} of the circular configuration shown in FIG. 5-1, transitioning from the circular shape 501a to the non-circular shape 501b, can provide a reduction in area/volume of the blood vessel, and therefore solutions that cause transitions between circular and non-circular blood vessel shapes between cardiac phases can provide compliance characteristics without the need for elasticity in the blood vessel wall tissue. For example, where a mechanism is implemented to cause a blood vessel to transition between circular and non-circular shapes in response to changing pressure conditions, such manipulation of the blood vessel shape can introduce volumetric change in the blood vessel in response to the typical changes in pressure experienced during the cardiac cycle, thereby increasing cardiac efficiency and reducing pulsatile load.

In view of the foregoing, examples of the present disclosure provide delivery systems for deploying stent implant devices and associated processes configured to transition the shape/area of a blood vessel from circular/more-circular to non-circular/less-circular shapes, and vice versa, to enhance compliance with respect to the area of the implant reshaping. Such stent implant devices/processes may affect vessel reshaping through dynamic reshaping of the structural shape of the stent in a way that produces a change in shape of the blood vessel in which it is implanted to produce a change in blood vessel area/volume between the systolic and diastolic phases of the cardiac cycle. The term “stent” is used herein in accordance with its broad and ordinary meaning and may refer to any device configured to be implanted in a lumen of a blood vessel, the device having a tubular form forming a lumen through which blood can flow.

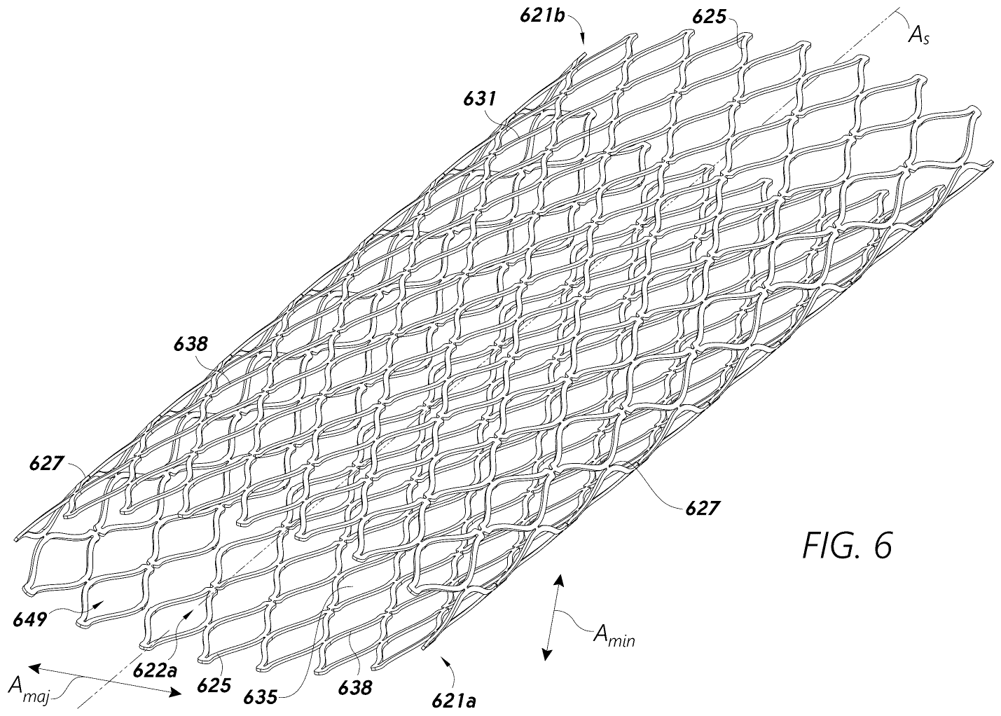

FIG. 6 shows a perspective view of a non-circular stent 600 in accordance with one or more examples. The stent 600 may be deployable within a blood vessel lumen using any delivery system example disclosed herein. The stent 600, as with other stents disclosed herein, may be formed of a tubular frame 631, which may form a wall around an axial channel 649, thereby defining the channel 649. As described herein, the frame wall 631 of the stent 600 can be considered a single, circumferentially-wrapped wall, or may be considered to comprise multiple walls, or wall segments. For example, with respect to oval stents and other non-circular stents, as illustrated in FIG. 6, such stents may be considered to comprise sidewall segments 625 that run along relatively long sides of the stent that are aligned generally with the orientation of the major axis/dimension Aₘₐⱼ of the stent, as well as end wall segments 627 on major-axis ends of the stent 600. The end walls 627 may be outwardly-curved/concave with respect to an axis Aₛ of the stent 600. The sidewalls 625 may be generally straight and/or less-curved compared to the end walls 627 over at least a portion of a length thereof, and/or may bow/deflect inward and/or outward, either in a resting, unpressurized state, or in conditions of hoop/wall stress on the frame 631. For example, the sidewalls 625 may bow outward such that the sidewalls 625 are concave from the perspective of the axis Aₛ of the stent 600.

Certain stent shapes are described herein, including non-circular-, oval-, peanut-, and other-shaped stents. It should be understood that such description of stent shapes refers to a shape of an axial cross-section of a stent. Although oval-shaped stents are described, it should be understood that the principles of the present disclosure may relate to stents having any non-circular shape in at least some configurations thereof (e.g., relaxed configuration). Descriptions of stents in a relaxed configuration should be understood to relate to a configuration that a stent naturally assumes in the absence of tension on the stent wall(s) from external forces (e.g., ambient fluid pressure, physical contact forces, etc.).

The stent frame 631 comprises stent wall(s) defining an elongated tubular structure having a first axial end 621a with a first opening 622a. The tubular structure may further comprise a second axial end 621b with a second opening 622b, wherein the lumen/channel 649 extends between the first opening 622a and the second opening 622b, traversing the length of the stent 600. The frame 631 and/or wall(s) thereof may comprise an open-cell structure adapted to be expanded to secure the stent 600 to a blood vessel internal (or external) wall, such as through a pressure-fit deployment, one or more tissue anchors/barbs, and/or endothelialization of the frame 631 to the vessel tissue over time.

The stent 600 may be elastically deformable between a first, non-circular configuration (e.g., configuration of stent 503 in FIG. 5-2) and a second, more-circular configuration (e.g., configuration of stent 503 in FIG. 5-1), with the stent 600 biased toward the first configuration. In some examples, the stent frame 631 may comprise a shape-memory and/or super-elastic material, such as nitinol. Although shown as an oval-shaped stent, the stent 600 may be any non-circular shape in a resting state thereof, such as a triangle, peanut, figure-8, and/or kidney shape.

The stent 600 may be configured to be percutaneously delivered to a blood vessel in a compressed delivery configuration. Once within the blood vessel lumen at the target deployment site, the stent 600 and/or frame 631 thereof may be configured to be radially expanded into direct surface contact with the blood vessel wall (e.g., the inner wall of an aorta segment). In some examples, the stent 600 may be configured to be expanded such that the perimeter of the stent 600 approximates and/or exceeds a perimeter of the blood vessel portion where the stent 600 is implanted, at least immediately prior to deployment/expansion of the stent.

In the oval configuration shown in FIG. 6, the stent 600 may have a cross-sectional area having a major/long-axis Aₘₐⱼ diameter that is substantially larger than the minor/short-axis Aₘᵢₙ diameter. For example, the major-axis diameter/dimension may be at least twice as long as the minor-axis diameter/dimension, or even 3, 4, 5, 6, or 7 times greater. The stent frame wall(s) 631 may be at least partially composed of struts 638 that form open cells 635 between the struts 638. The dimensions and/or shape of the stent 600 may vary based on the particular application and/or target implantation anatomy. The stent 600 may have a length of between 1-45 cm.

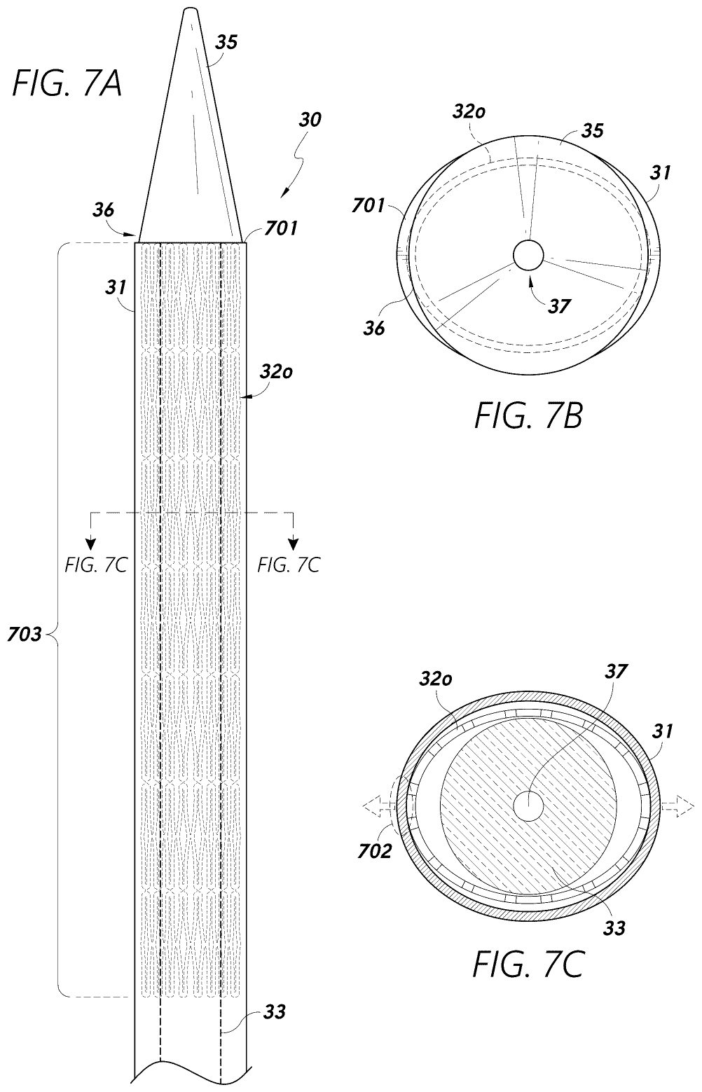

Delivery of non-circular stents using traditional delivery systems can present various challenges. For example, the various compressed and/or expanded dimensions of non-circular (e.g., oval) stents can cause certain issues relating to delivery system advancement and/or retrieval. Some such issues can be understood with respect to the example delivery systems and stents illustrated in FIGS. 7-10.

FIGS. 7A-7C show side, axial, and axial cross-sectional views, respectively, of a delivery system 30 having a non-circular stent 32o disposed therein in accordance with one or more examples. The delivery system 30 can comprise one or more catheters or sheaths 31 used to advance and/or deploy the non-circular stent implant device 32o, which may be disposed at least partially within the delivery system 30 during portions of a transcatheter delivery process. The terms “capsule,” “sheath,” “catheter,” “shaft,” “lumen,” and the like are used herein according to their broad and ordinary meanings, and may refer to any tubular structure or component forming an axial/longitudinal channel or lumen therein. In some contexts, an outer sheath of a delivery system may be referred to as a ‘capsule.’ Alternatively, such outer sheath may be referred to as a ‘catheter’ or ‘shaft,’ or simply a ‘sheath.’ With respect to delivery systems having multiple shafts/sheaths configured to move axially relative to one another, wherein one such sheath/shaft is disposed within a channel/lumen of the other, the outer most shaft/sheath of such assembly may be referred to as a ‘capsule’ to connote encapsulation by such components of one or more internal components. When an outer sheath is moved proximally relative to nose cone/guidewire shaft, such action may be referred to as unsheathing of the nose cone shaft and/or implant device coupled and/or disposed thereon. In some implementations, the delivery system 30 may be advanced to the target anatomical site through an introducer sheath.

The delivery system 30 may include an elongate shaft including a distal end, which is shown in FIGS. 7A-7C, wherein the shaft may be coupled at a proximal end thereof to a housing in the form of a handle, for example. The handle (not shown in FIG. 7A for visual clarity) may be configured for manual manipulation when operating the delivery system 30. The delivery system 30 may be configured to be inserted into a patient's body, such as into/within the patient's vasculature, and advanced/directed to a target treatment site. Such insertion may be percutaneous and minimally invasive, such as through transfemoral or other venous or arterial entry.

The distal portion 703 of the delivery system 30 may serve as an implant retention assembly, wherein an implant and/or other component(s) of the delivery system may be covered by an outermost sheath 31 to form a capsule. The implant retention portion 703 may be configured to retain the implant 32o until the desired time for deployment of the implant. The delivery system 30 may be inserted into the patient's body and navigated to the desired deployment location to position the distal end of the delivery system 30 at the target implantation site. The delivery system 30 may then be operated to deploy the implant 32o from the distal portion of the system 30. A deflection mechanism may be provided that operates to actively deflect at least a portion of the elongate shaft aspect (e.g., assembly of the elongate sheath 31, nose cone shaft 33, and/or other component(s)) of the delivery system 30, such as through the tensioning of one or more pull wires or the like.

The delivery system 30 may include a nose cone 35 at and/or associated with the distal end/portion of the elongate shaft 24. The nose cone may form the tip of the delivery system 30 when transporting the implant 32o. The nose cone 35 may advantageously present an atraumatic interface for the distal end of the delivery system. For example, the nose cone 35 may be pliable/flexible to reduce the risk of injury to the patient anatomy when contacted by the tip of the delivery system. The nose cone 35 may have a tapered shape from its proximal end/base 36 to its distal end/tip. The nose cone 35 may facilitate advancement of the distal end of the delivery system 30 through the tortuous anatomy of the patient and/or an outer delivery sheath or other conduit/path.

The nose cone 35 may be coupled to the delivery system via a shaft 33, which may be coupled to and/or integrated with the base 36 of the nose cone. The shaft 33 can be disposed at least partially within the outer sheath 31 and configured to be axially advanced relative to the sheath 31, thereby causing the nose cone 35, shaft 33 distal portion, and implant 32o to advance distally from the distal end of the sheath 31. For example, the operation of the delivery system 31 for deployment of the stent 32o may involve advancing the shaft 33 distally and/or retracting the sheath 31 proximally to thereby cause the shaft 33 and implant 32o to pass through a distal opening of the sheath 31 to permit deployment of the implant 32o outside of the sheath 31. The shaft 33 may comprise a straight shaft and/or may include various features for holding the implant 32o in-place during delivery. In some implementations, the shaft 33 includes an adapter component configured to provide an expanded diameter of the shaft 33 to hold the implant 32o. The shaft 33 and other similar delivery system components described herein may be referred to as a nose cone shaft.

In some implementations, the delivery system 30 may optionally comprise a pusher shaft (not shown), which may be slidingly disposed within the outer sheath 31 proximal and/or adjacent to the implant device 32o. Such a pusher may be coupled to or integrated with the nose cone shaft 33, or may be configured to slidingly pass over the shaft in some examples. Pusher components, where implemented, can be used to push/advance the implant 32o and/or nose cone 35 relative to the outer shaft/sheath 31 as a means to deploy the device 32o from the sheath 31. In some examples, a pusher or component of the shaft 33 is releasably attached to the frame of the stent 32o and/or other component(s) of the implant device 32o, wherein after the device 32o has been deployed from the sheath 740, positioned in the desired implantation site/position, and/or expanded, the delivery system may be disengaged from the implant device 32o to release the device 730 and allow for removal/withdrawal of the delivery system 30. For example, the shaft 33 or other component(s) of the delivery system 30 may comprise one or more feet, arms, tabs, or the like. The implant device 32o and/or other component(s) of the delivery system 30 may comprise one or more radiopaque markers that may be referenced/imaged to determine/confirm the position of the implant device 32o and/or delivery system 30 at various stage(s) of the implantation process using a suitable imaging modality. In the compressed delivery configuration, the stent 32o may be somewhat elongated compared to a fully-expanded configuration thereof due to at least some of the struts/cells of the frame of the stent being deflected into more longitudinally-oriented configurations when radially crimped/compressed.

In some implementations, the nose cone 35 may have a circular base, as shown in FIG. 7B. It may be desirable for the nose cone base 36 to have a shape that conforms to and/or is otherwise associated with the cross-sectional shape of the sheath 31, such as circular with respect to the illustrated configurations of the delivery sheath/capsule 31 and the nose cone 35. For example, by matching the shape of the nose cone base 36 to that of the circular delivery sheath 31, the nose cone 35 may effectively cover at least portions of the distal end/edges of the sheath from contact with the patient anatomy during delivery, at least in the absence of deformation of the sheath 31 as described in greater detail below.

The delivery system 30 may further be configured to have a guidewire disposed at least partially within the delivery system 30 and/or coupled thereto in a manner to allow the delivery system 30 to follow a path defined by the guidewire. In some implementations, a guidewire may pass through an interior lumen/channel 37 of the implant device and/or through a lumen of a pusher device or tube of the delivery system 30. In some implementations, the nose cone shaft 33 may be, or may be disposed around/over, an innermost component of the delivery system 30 that comprises/forms the inner guidewire lumen 37. The guidewire lumen 37 may extend for the length of the elongate portion of the delivery system 30.

When a circular sheath is utilized for delivery of a non-circular stent, such as an oval stent or other non-circular implant device, the non-circular/oval shape of the implant device may exert certain forces on the inner diameter of the sheath that can deform the sheath/capsule in which the implant device is retained during delivery of the implant device. For example, with respect to the implementation of FIGS. 7A-7C, the outer sheath 31 of the delivery system 30 may have a circular axial cross-section in a natural shape thereof. For delivery, the implant device 32o, while having a natural oval shape, may be compressed to a generally circular cross-sectional delivery configuration to fit inside the sheath 31. However, while the stent 32o may be radially compressed to a more circular shape compared to the biased oval or other non-circular relaxed shape thereof, the non-circular biasing of the stent frame 32o may cause at least slight deformation of the cross-sectional shape of the sheath. For example, FIGS. 7A-7C show slight major-axis expansion/ovalization of the sheath 31 in the distal portion 703 thereof due to major-axis forces of the crimped stent 32o on opposite sides 702 of the sheath 31.

The deformation/ovalization of the sheath 31 by the compressed oval stent 32o can result in exposed distal edges/ridges/lips 701 protrude radially outside of the axial profile of the circular nose cone base 36, wherein such edges 701 can present traumatic contacts with respect to the anatomy through which the delivery system is advanced. Such contact surfaces 701 can produce abrasions or other damage to the patient anatomy. Furthermore, deformation/ovalization of the otherwise-circular delivery sheath/capsule 31 can cause damage to the sheath 31 and/or other component(s) of the system.

In addition to concerns relating to the deformation of the delivery sheath by a non-circular stent implant, the delivery/deployment of non-circular implants and/or other implant devices having at least one relatively narrow dimension (e.g., a dimension having length/distance that is less than a minimum diameter of the delivery sheath/capsule and/or nose cone base) can present certain other issues, such as with respect to nose cone retrieval after implant deployment.

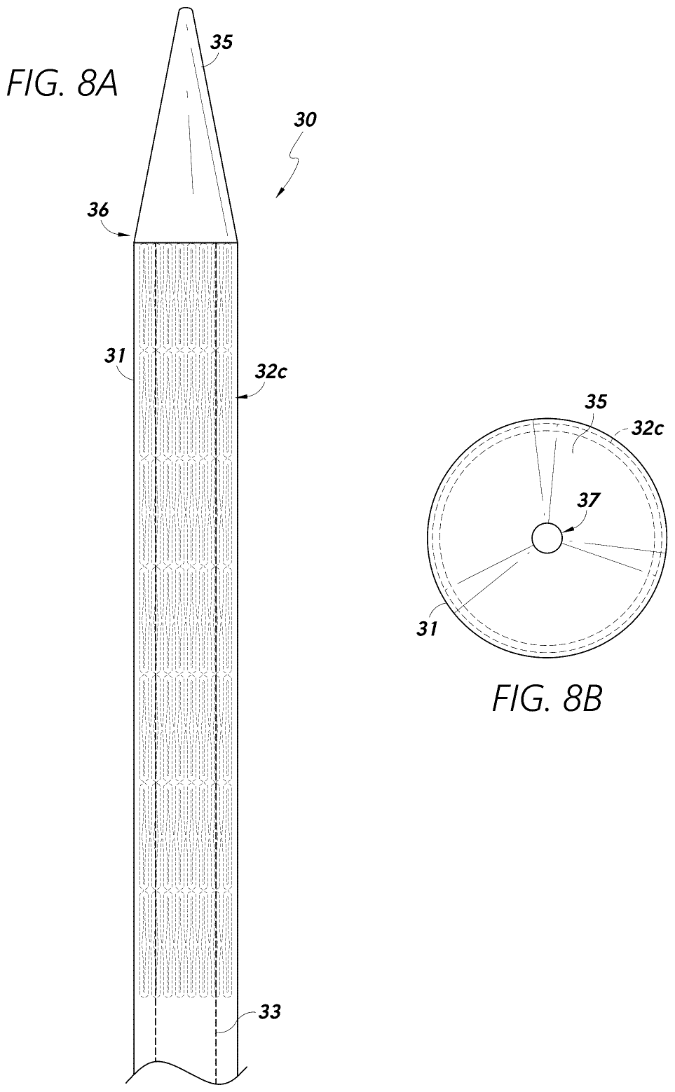

FIGS. 8A and 8B show side and axial views, respectively, of a delivery system 30 having a circular stent 32c disposed therein in accordance with one or more examples. The delivery system 30 shown in FIGS. 8A and 8B can be similar to the delivery system described above in connection with FIGS. 7A-7C. For example, the outer sheath/shaft 31 of the delivery system 30 shown in FIGS. 8A and 8B can have a circular axial cross-section, as with the base 36 of the nose cone 35.

The delivery system 30 of FIGS. 8A and 8B may be utilized to deliver the implant device 32c, such as a stent or similar device configured to expand when deployed from the delivery system 30. The nose cone 35 of the delivery system 30 is coupled to a nose cone shaft 33, which may serve as, or be disposed about, a guidewire lumen 37 and/or otherwise provide a channel through which a guidewire may slidingly be disposed. Unlike the non-circular/oval stent/implant 32o shown in FIGS. 7A-7C, the implant device 32c may be a stent or the implant device having a generally-circular cross-sectional shape, such that expansion thereof produces a shape having a minimum inner diameter that is greater than the outer diameter dₙ of the base 36 of the nose cone 35 and/or the delivery system sheath.

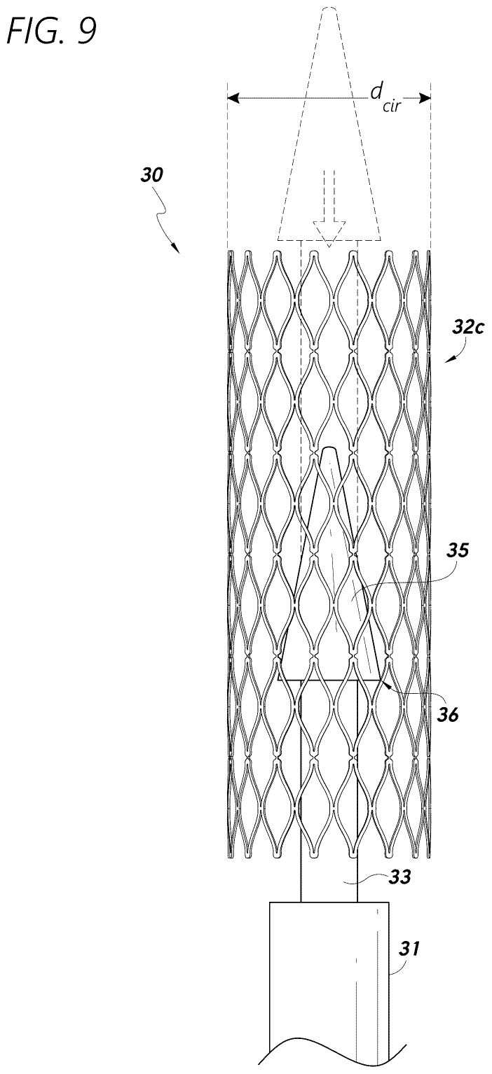

FIG. 9 shows the delivery system 30 in a nose-cone-retrieval configuration associated with deployment of the stent implant 32c in accordance with one or more examples. The process of deploying the stent implant may involve first distally advancing the nose cone 35, and with it the nose cone shaft 33, relative to the outer sheath 31. The stent implant 32c may be crimped and disposed on the nose cone shaft 33, on a distal portion thereof, during delivery. The portion of the shaft 33 on which the stent 32c is mounted in the delivery system 30 may comprise the guidewire lumen, and/or may comprise an adapter structure/component coupled on/to the guidewire lumen, such adapter having a shape configured to accommodate/hold the crimped stent implant 32c. For example, an adapter component may be over-molded on a relatively thin guidewire lumen/shaft, wherein such adapter and guidewire lumen may be collectively referred to herein as the nose cone shaft or guidewire lumen/shaft.

In some implementations, in order to deploy the stent 32c, the outer sheath 31 may be pulled proximally, wherein the stent 32c may be held relative to the nose cone 35 and nose cone shaft 33 by a proximal pusher, and/or by mechanical coupling to the nose cone shaft 33 and/or other component(s) of the delivery system 30. When the sheath 31 is retracted a sufficient distance to expose the stent 32c distal of the sheath 31, the stent 32c may be permitted to expand to a diameter d_{cir} greater than that of the delivery system sheath 31 and/or a nose cone base 36. For example, such expansion may be implemented using a balloon catheter or other expansion device, and/or the stent frame 32c may have a shape-memory/superelasticity characteristics designed to cause expansion of the frame when released from the mechanical constraints of the outer sheath 31.

With the nose cone 35 and nose cone shaft 33 distally advanced to expose and deploy the stent 32c, the stent 32c may expand radially away from the nose cone shaft 33, such that the stent 32c is not coupled to the shaft 33 and can be implanted in the anatomy (e.g., aorta) independently of the delivery system 30. With the stent 32c expanded, proximal retraction/withdrawal of the nose cone shaft 33 may pull the nose cone 35 back through the inner lumen/channel of the implant device 32c to thereby allow for removal of the nose cone 35 and delivery system 30 while maintaining the implant 32c in-place. For example, the nose cone 35 may be proximally drawn back towards and/or at least partially into the outer sheath 31, and/or the sheath 31 and nose cone 35 may be pulled together proximally back through the access path and out of the patient's body.

FIG. 9 shows the nose cone 35 being drawn proximally through the channel of the deployed/expanded stent 32c for retrieval thereof. The expanded inner diameter of the stent 32c can provide a channel through the stent that is sufficiently wide to facilitate the passage therethrough of the nose cone base 36 without getting caught on the stent frame, thereby facilitating removal of the delivery system 30. The stent 32c, having a circular cross-sectional shape, may have an approximately constant diameter d_{cir} at various angular positions. That is, unlike certain oval-shaped stents as described herein, which may have major- and minor-axis diameters of different dimensions, the circular stent 32c, as expanded, may not have a substantially reduced minor axis dimension. For implant devices having relatively narrow minor-axis dimensions, such reduced dimensions can interfere with the ability of the nose cone base 36 to be proximally drawn through the inner diameter of the stent where the nose cone 35 and/or delivery system capsule/sheath 31 have a circular cross-sectional shape.

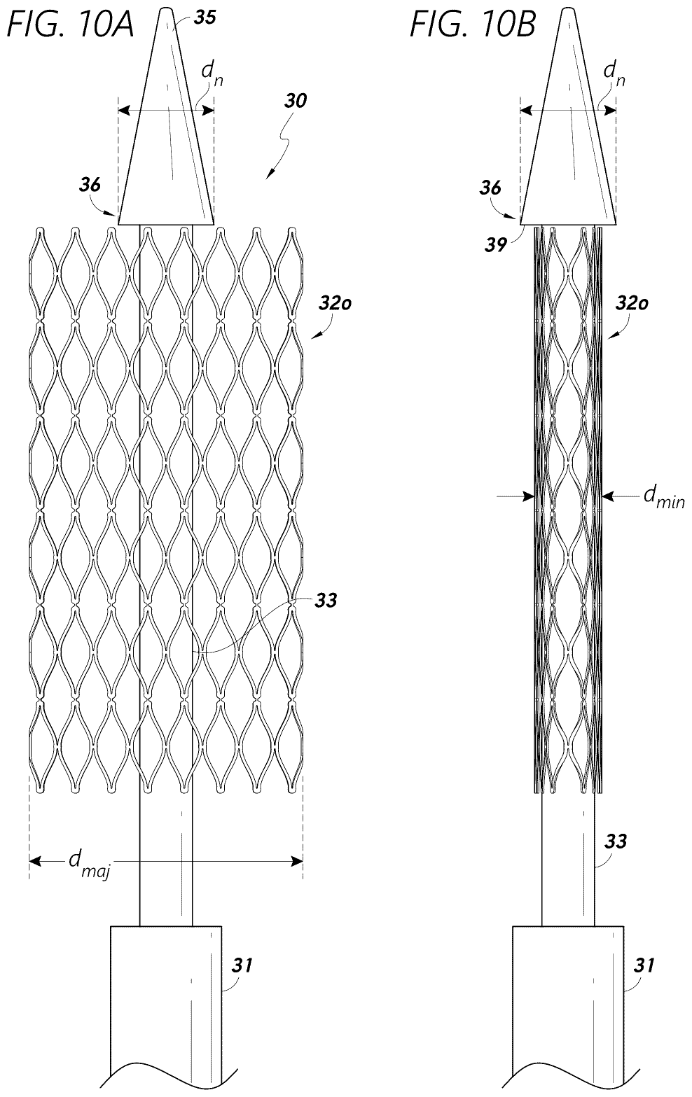

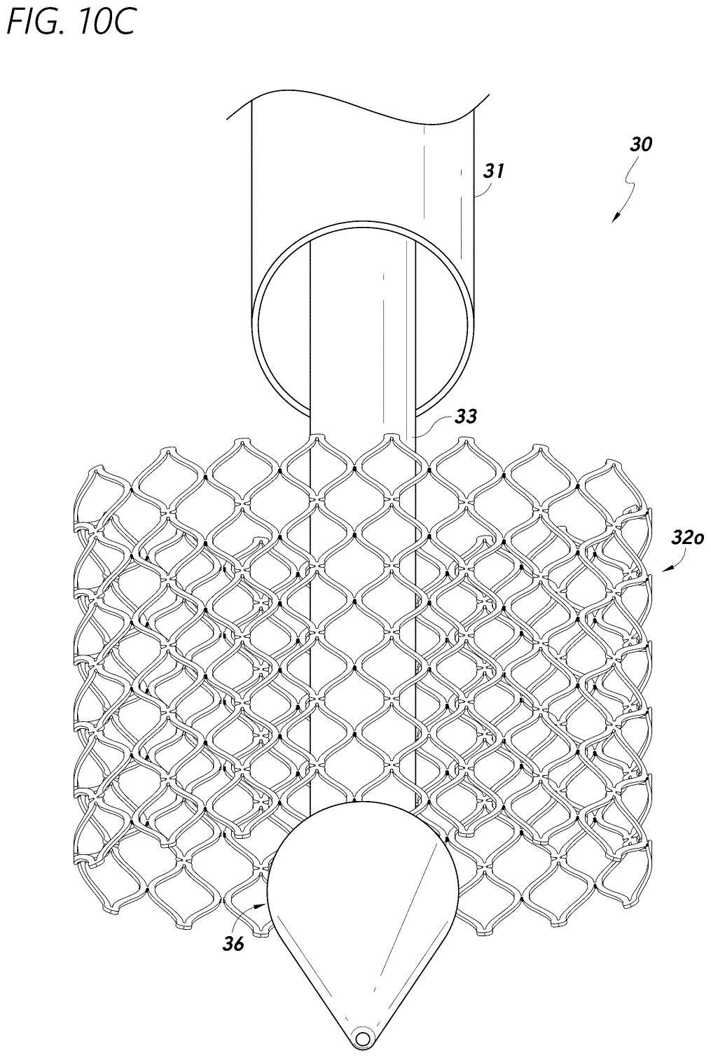

FIGS. 10A-10C show a delivery system 30 as used to deploy a non-circular/oval stent 32o, wherein a nose cone 35 of the delivery system 30 has a diameter dₙ that is greater than a minor-axis diameter dₘᵢₙ of the non-circular stent 32o in accordance with one or more examples. The delivery system 30 shown may have features similar to those described above in connection with FIGS. 8A and 8B. For example, the nose cone 35 of the delivery system 30 may have a circular base 36. The delivery system 30 is configured to transport, within an outer sheath 31 thereof, the non-circular stent 32o, which is configured to be expanded to an oval or other non-circular shape when deployed from the sheath 31. The axial cross-sectional shape of the stent 32o, when expanded, presents a major-axis dimension dₘₐⱼ and a minor-axis dimension dₘᵢₙ that is substantially less than the major-axis dimension dₘₐⱼ. The stent 32o, during delivery, may be disposed on/about the nose cone shaft 33, which may have a diameter that is less than that of the base 36 of the nose cone 35.

FIGS. 10A-10C show the delivery system 30 with the nose cone shaft 33 and the nose cone 35 extended distally from the distal end of the sheath 31, such that a sufficient portion of the nose cone shaft 33 is exposed from the sheath 31 to fully deploy the stent 32o from the sheath 31. As expanded and in the biased oval/diastolic configuration, the major-axis dimension dₘₐⱼ of the stent 32o may be between 1-4 cm (or larger/smaller depending on the particular anatomy), and the minor-axis dimension dₘᵢₙ may be between 20-50 percent of the major-axis dimension dₘₐⱼ. However, other sizes and/or shapes are also within the scope of this disclosure.

When delivered to the target anatomical site, the stent 32o may be contained within the outer sheath 31 in a compressed/crimped delivery configuration, wherein the stent 32o may have a more-circular axial cross-sectional shape in the crimped delivery configuration thereof compared to the expanded oval shape shown in FIGS. 10A-10C. In some implementations, when the stent 32o is deployed, the minor-axis dimension of the stent 32o may reduce to and/or remain at a dimension dₘᵢₙ that is less than the diameter dₙ of the circular base 36 of the nose cone 35. In such instances, the circular nose cone base 36, having a diameter dₙ greater than at least one diameter/dimension of the implant device 32o deployed between the nose cone 35 and the distal end of the outer sheath 31, may encounter obstruction/interference on a proximal surface/edge 39 thereof when attempting to draw the nose cone 35 proximally back through the lumen/channel of the implant device 32o.

As shown in the configuration of FIGS. 10A-10C, the maximum outer diameter dₙ of the nose cone base 36 can be greater than the minor-axis dimension dₘᵢₙ of the stent implant device 32o, which can prevent or impede retrieval of the nose cone 35 through the stent 32o after deployment. Therefore, with respect to non-circular stents and similar implant devices, delivery systems having circular sheath and/or nose cone features, wherein the nose cone has a diameter approximately equal to or greater than that of the circular sheath, implant deployment/delivery using such delivery systems may result in damage inefficiencies and/or damage to patients, implants, and/or delivery system components.

### Non-Circular Delivery System Components

Examples of the present disclosure provide for delivery system components that have non-circular axial shape, such as sheath and/or nose cone components having non-circular (e.g., oval) shapes, or other shapes that provide a relatively narrower dimension/diameter that better conforms to the channel shape of a non-circular/oval implant device. As described above, where oval or other non-circular stents or implant devices are delivered using circular sheaths, certain exposed lips/edges can form in the sheath due to deformation from mechanical forces of the implant device on the inner diameter of the sheath, wherein such lips/edges can cause tissue abrasion. Furthermore, circular nose cones can be difficult or impossible to withdraw through the channel of an implant device having a relatively narrow channel inner diameter dimension. Some examples of the present disclosure alleviate such issues by providing nose cone and delivery system components that have diameters/dimensions that accommodate non-circular stent/implant delivery and/or deployment.

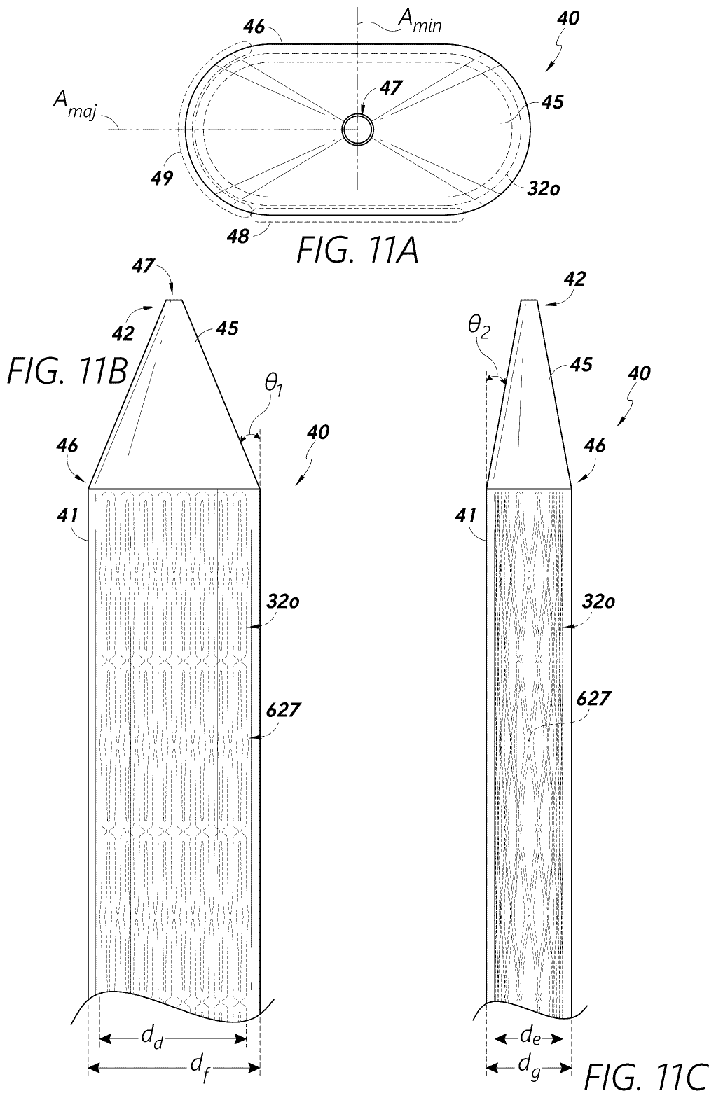

FIGS. 11A-11C show axial, major-axis side, and minor-axis side views, respectively, of a non-circular delivery system 40 in accordance with one or more examples. The delivery system 40 is adapted for delivering non-circular stents and/or similar implant devices, and may include an oval or other non-circular-shaped, at least partially rigid capsule/sheath 41. Additionally or alternatively, the delivery system 40 may comprise a nose cone 45 that has a non-circular (e.g., oval) base 46. The nose cone base 46 may be the same shape as the sheath 41, at least with respect to a distal end of the sheath 41, such that the nose cone 45 can come into flush engagement with the sheath 41.

The delivery system sheath/capsule 41, as shown in the axial view of FIG. 11A, has an oval or other non-circular axial cross-sectional shape, which may conform more closely to the natural cross-sectional shape of an oval stent or other non-circular implant device 32o delivered in the delivery system. The sheath may comprise relatively material, such as plastic, polymer, and/or the like. Due to the oval/non-circular shape of the sheath 41, relative to a circular delivery system sheath, the delivery system 40 may provide a reduction in deployment forces at the major-axis sides/ends 627 of the implant device 32o against the sheath 41 inner diameter. The sheath 41 may be manufactured using relatively low-friction materials to further facilitate advancement of the sheath 41 through the tortuous anatomy of the patient's vasculature and/or an introducer sheath.

The cross-sectional shape of the sheath 41 may conform to and/or be matched to that of the base 46 of the nose cone 45, thereby providing a relatively smooth transition between the nose cone 45 and the sheath 41 without edges or surfaces of the distal end of the sheath 41 jutting out radially beyond the profile of the nose cone base 46. The sheath 41 and/or nose cone base 46 may be considered oval-shaped with respect to the shape of the axial cross-section thereof, as shown in FIG. 11A. The term “oval” is used herein according to its broad and ordinary meaning and may be used substantially interchangeably with the term “ellipse” and/or “oblong,” which terms are likewise used according to their broad and ordinary meanings. The term “oval” may be used to refer to any non-circular closed curve having major and minor axes, the major axis being greater than the minor axis. With respect to “oval”-shaped delivery system components disclosed herein, such components may have relatively flatter minor-axis sides/sidewalls 47 (compared to curved major-axis ends/sidewalls 49), wherein the sides 48 may or may not bow radially outwardly. Major-axis sides of sheath and/or nose cone components as described herein may be identified as sides/portions that are intersected by a major axis Aₘₐⱼ of the delivery system that runs through an axial center of the sheath 41 and/or nose cone 45. Minor-axis sides of such delivery system components may be identified as sides/portions that are intersected by a minor axis Aₘᵢₙ of the delivery system that runs through the axial center of the sheath 41 and/or nose cone 45. The description below of the various examples of delivery system components having non-circular cross-sectional portions/sections provide further context for interpreting the terms “oval” and “non-circular” in the context of oval delivery system components having oval portions/segments. Example delivery system components of the present disclosure may be considered to have an oval shape whether or not the shape thereof is definable by an algebraic curve. Example delivery system components of the present disclosure may be considered oval-shaped when the sides thereof in an axial-cross-sectional perspective form(s) a closed curve in a plane that is non-circular; one or more segments/areas thereof may resemble the outline of a portion of an egg. Oval delivery system components of the present disclosure may include either one or two axes of symmetry of an ellipse, such as the illustrated major Aₘₐⱼ and minor Aₘᵢₙ axes. The axial cross-section of some examples of oval delivery system components of the present disclosure may resemble the union of two semicircles on opposite sides of a rectangle, providing a shape evoking the likeness of a speed skating rink or an athletics track. In some contexts, oval delivery system components may be referred to as having a “stadium” or elongated oval shape.

The sheath 41 and/or nose cone base 46 may have a minor-axis dimension d_{g} that is substantially less than a major-axis dimension d_{f} thereof, as illustrated. For example, the major-axis dimension d_{f} may be at least twice that of the minor-axis dimension d_{g} in some examples. The nose cone 45 may be tapered in continuous major-axis θ₁ and minor-axis θ₂ taper angles from the base 46 of the nose cone 45 to the tip 42 thereof, as shown in FIGS. 11A-11C, or the nose cone 45 may have different angles of taper moving from the base to the tip, as described in greater detail below. In the example of FIGS. 11A-11C, the taper angle may be constant for a given circumferential/perimeter portion moving in the axial direction with respect to the axis of the delivery system. However, due to the shape of the base 46 of the nose cone 45, the taper of the nose cone 45 may have different angles relative to the axis of the nose cone 45 on the minor-axis side (see FIG. 11C) of the nose cone 45 than on the major-axis side (see FIG. 11B). That is, the angle θ₂ on the minor-axis side may be less than the angle θ₁ on the major-axis side, as illustrated. In some implementations, the major-axis taper angle θ₁ may be between 15-30°, such as about 23°, whereas the minor-axis taper angle θ₂ may be between 5-15°, such as about 10°.

The use of an oval or other non-circular delivery system sheath may provide various advantages. For example, the non-circular shape of the sheath 41 may provide improved visibility of the delivery system during delivery using an imaging modality intraoperatively, such as x-ray, fluoroscopy, ultrasound, or the like. For example, the visually identifiable shape of the sheath 41 may provide visibility with respect to the major and minor axes thereof, wherein identification of such axes/dimensions of the delivery system can inform the surgeon/technician regarding the roll/orientation of the sheath 41 in the anatomy. Therefore, with respect to implant deployment procedures using oval delivery system components, improved visibility provided by the delivery system shape can improve outcomes.

Due to the cross-sectional shape of the sheath 41 better conforming to the shape of an oval stent implant 32o disposed therein, the sheath may produce reduced stresses on the crimped/compressed implant device, particularly with respect to major axis ends/sides thereof. That is, the shape of the sheath may better conform to the natural shape of the implant device, and therefore less forces/pressures may be imposed on the implant device by the sheath to hold the implant in the current pressed configuration. Therefore, the non-circular shape of the delivery system sheath 41 can help maintain the structural integrity of the implant device 32o by not requiring compression thereof to a circular crimped shape. Rather, the stent/implant 32o can be transported in a delivery configuration that allows for a greater major-axis dimension d_{d} of the implant relative to a narrower minor-axis dimension dₑ thereof, thereby requiring less structural stress from deviation from the biased non-circular shape of the implant 32o during delivery.

Due to the oval/non-circular shape of the sheath 41, the delivery system 40 may be at least partially restricted with respect to bending orientation thereof. For example, the delivery system 40 may be configured to bend/articulate more readily and/or with reduced resistance in the dimension of the minor axis Aₘᵢₙ of the sheath 41, whereas articulation/deflection in the major-axis plane Aₘₐⱼ may present greater resistance. Therefore, surgical procedures utilizing delivery systems similar to that shown in FIGS. 11A-11C can require the surgeon to be cognizant of delivery system orientation, such that the orientation of the delivery system may be controlled/manipulated to align the minor axis of the sheath 41 with curves/bends in the access path followed to arrive at the target site within the vasculature or other anatomy. In some implementations, at least a portion of the delivery system sheath 41, such as an articulated oval portion thereof, may be formed at least partially of two or more disjointed links, which may comprise one or more struts or other mechanical features configured to allow relative articulation/deflection of the links relative to one another, which may facilitate articulation in the major-axis dimension Aₘₐⱼ as well as the minor-axis dimension Aₘᵢₙ. The reduced minor-axis dimension d_{g} of the oval/non-circular sheath 41 can improve/increase the bending radius of the sheath 41 relative to certain circular sheaths as described above.

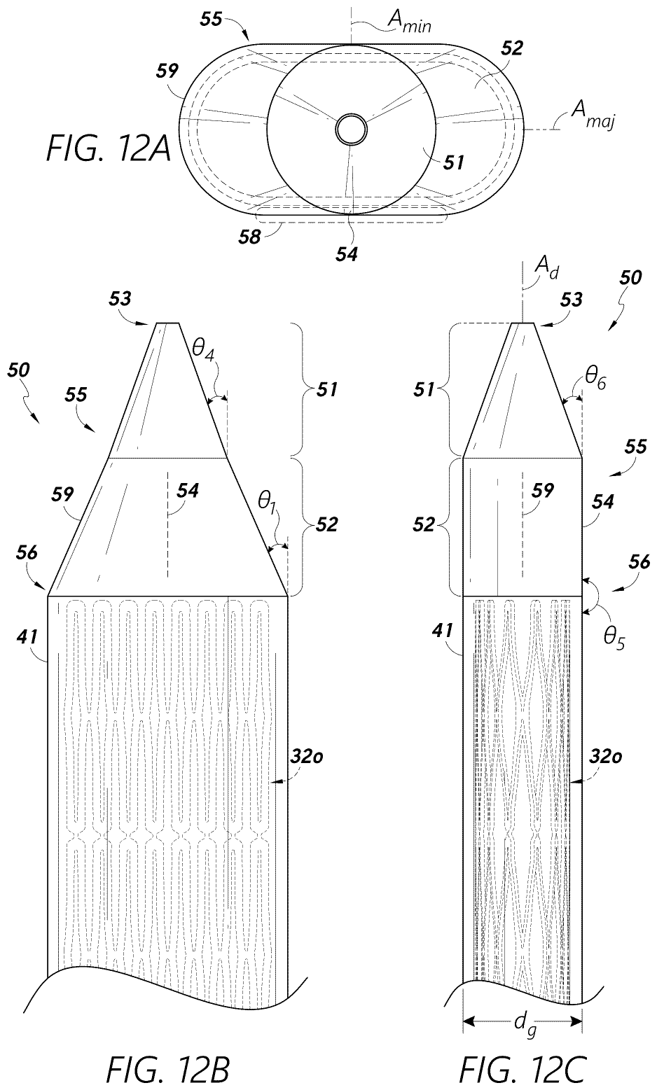

FIGS. 12A-12C show axial, major-axis side, and minor-axis side views, respectively, of a non-circular, transitional nose cone 55 in accordance with one or more examples. The nose cone 45 described above in connection with FIGS. 11A-11C is presented as having constant taper angles between the base and tip of the nose cone; the nose cone of FIGS. 12A-12C provide an alternative nose cone implementation for a nose cone of a delivery system having a non-circular axial shape. The nose cone 55 of FIGS. 12A-12C provides a nose cone having transitional tapered side(s)/portion(s), wherein a taper/angle on one or more sides or portions of the nose cone circumference/perimeter transitions from a first angle (e.g., angles θ₃, θ₅) to at least a second angle (e.g., angles θ₄, θ₆) moving from the base 56 to the tip 53 of the nose cone 55. The transition between taper angles moving in the axial dimension A_{d} delineate distal/tip 51 and proximal/base 52 nose cone portions.

As an example, the nose cone 55 may include a circular tip distal portion 51, wherein the taper θ₄, θ₆ of such portion of the nose cone may be constant moving around the circumference of the nose cone 55 relative to the axis A_{d} of the nose cone 55. That is, the distal portion 51 of the nose cone may provide a circular cone shape, rather than the oval shape of the distal portion of the nose cone 45 shown in FIGS. 11A-11C. However, as the nose cone can advantageously be designed to match and/or cover the distal end of the oval/non-circular sheath 41, it may be necessary or desirable to transition from the circular shape of the distal portion 51 of the nose cone 55 to the oval/non-circular shape associated with the distal end of the sheath 41 at the base 56 of the nose cone 55. Therefore, the proximal portion 52 of the nose cone 55 may have a taper that has a greater angle θ₃ at major-axis sides 58 (e.g., at the intersection of the sides 58 and the minor axis Aₘᵢₙ) and a lesser angle θ₅ at the minor-axis sides 59 (e.g., at the intersection of the sides 59 and the major axis Aₘₐⱼ). In some implementations, at the minor-axis Aₘᵢₙ, the proximal portion 52 of the nose cone 55 may not be tapered, but rather may be substantially parallel with respect to the wall(s) thereof relative to the nose cone axis A_{d}. That is, the circular portion 51 of the nose cone 55 may have a diameter equal to the minor-axis diameter d_{g} of the oval base 56 of the nose cone 55, such that the sidewalls 54 of the nose cone 55 are substantially straight/parallel on the minor-axis sides, as shown in FIG. 12C. Alternatively, some amount of taper may be implemented in the minor-axis sidewalls 54, wherein such taper angle may be an angle less than the angle θ₃ of taper at the major-axis sidewalls 59.

The taper angle θ₃ at the major-axis sides 59 of the proximal portion 52 of the nose cone 55 may match the taper angle θ₄ of the circular distal portion 51 of the nose cone 55, or may have a taper angle less than or greater than the taper angle θ₄ of the distal nose cone portion. For example, as illustrated in FIG. 12B, deflection of the proximal portion 52 of the nose cone 55 at the major-axis ends/sides 59 may be slightly greater than the angle θ₄ of taper/deflection of the distal portion 51 of the nose cone with respect to the same circumferential/perimeter position thereof.

The more-circular shape of the distal portion 51 of nose cone 55 may facilitate advancement of the nose cone 55 through the patient's vasculature and/or into an introducer device/port, which may be designed to receive generally-circular delivery system components. The oval/non-circular proximal portion 52 of the nose cone 55, on the other hand, may facilitate relatively easier retrieval of the nose cone 55 through an implant device channel/lumen after deployment of the implant device by presenting a nose cone dimension de that is reduced relative to certain circular nose cones of delivery systems having different dimensions. The circular shape of the distal portion 51 of the nose cone 55 can facilitate mating of the delivery system 50 with sheath access components in a manner as to reduce the risk of blood/fluid leakage through introducer components. That is, the circular tip 51 may provide for an initial insertion of the delivery system 50 that conforms in a desirable manner to the configuration of the introducer component(s).

### Modifiable Nose Cones

The retrieval of delivery system nose cone components can be facilitated through the use of nose cones that are configured to change configuration between wider and narrower radial profiles. That is, according to some examples of the present disclosure, a nose cone may be transition from a relatively wider profile to relatively narrow profile, wherein the reduced profile allows for the nose cone to more easily pass through the channel/lumen of a deployed implant device, such as an oval/non-circular stent.

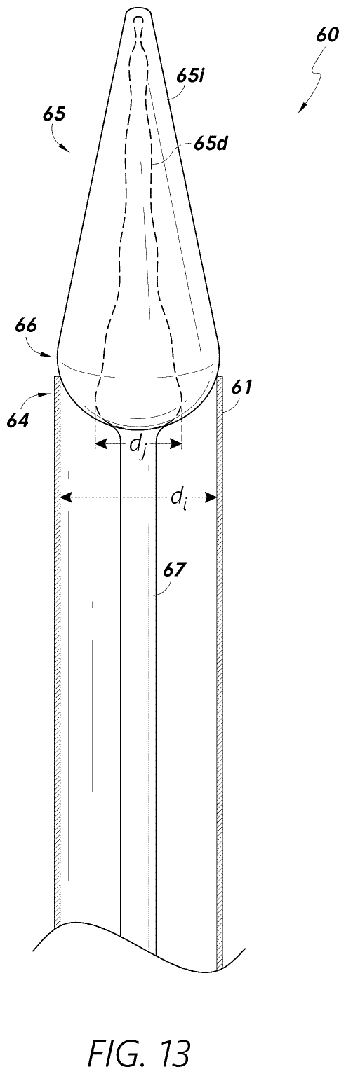

FIG. 13 shows a side view of a delivery system 60 including an inflatable nose cone 65 in accordance with one or more examples. FIG. 13 shows the nose cone 65 disposed at a distal end 64 of a delivery system sheath 61, wherein the nose cone 65 is shown in an inflated 65i configuration in which the nose cone 65 has a diameter/dimension dᵢ that is approximately equal to or greater than the diameter of the delivery system sheath 61, such that the nose cone 65 can cover the distal end 64 of the sheath 61 and prevent abrasion or catching thereof on the patient anatomy during delivery.

FIG. 13 further shows in dashed-line the nose cone 65 in a deflated 65d state having a reduced diameter. Inflation and/or deflation of the nose cone 65 may be implemented through a channel/lumen 67 through which air or other gas or fluid may be withdrawn and/or inflated with respect to the nose cone structure 65. The nose cone 65 may comprise fluid-tight material, such that fluid introduced into the nose cone 65 form can cause inflation/expansion thereof, whereas aspiration/withdrawal of fluid from the balloon 65 through the lumen 67 can cause deflation thereof. The fluid used for nose cone inflation may comprise saline solution or other liquid, or carbon dioxide gas or other gas. The fluid channel/lumen 67 may pass from the base 66 of the nose cone through the elongate shaft/sheath 61 of the delivery system to a proximal reservoir or other component configured to receive and/or provide fluid between the nose cone 65 and the reservoir. Use of saline solution or other liquid may be desirable due to the incompressibility of such media. Furthermore, saline solution may provide reduced risk of injury due to rupture of the nose cone compared to certain gaseous solutions.

The inflation tube 67 can advantageously be at least partially rigid to thereby maintain a good pathway through which fluid can be provided/withdrawn. In some implementations, the lumen/channel 67 comprises one or more one-way or two-way valves to control the transfer of fluid therethrough, such as for the purpose of preventing premature deflation of the nose cone. The fluid channel 67 can serve as and/or be formed by a nose cone shaft or adapter component/shaft configured to have disposed thereabout an implant device, such as a stent, as described below in connection with the process 1400. The balloon nose cone 65 may have an axial channel therethrough, wherein the inflatable portion(s) of the nose cone 65 are disposed around/about the channel, wherein the channel may serve as a guidewire channel.

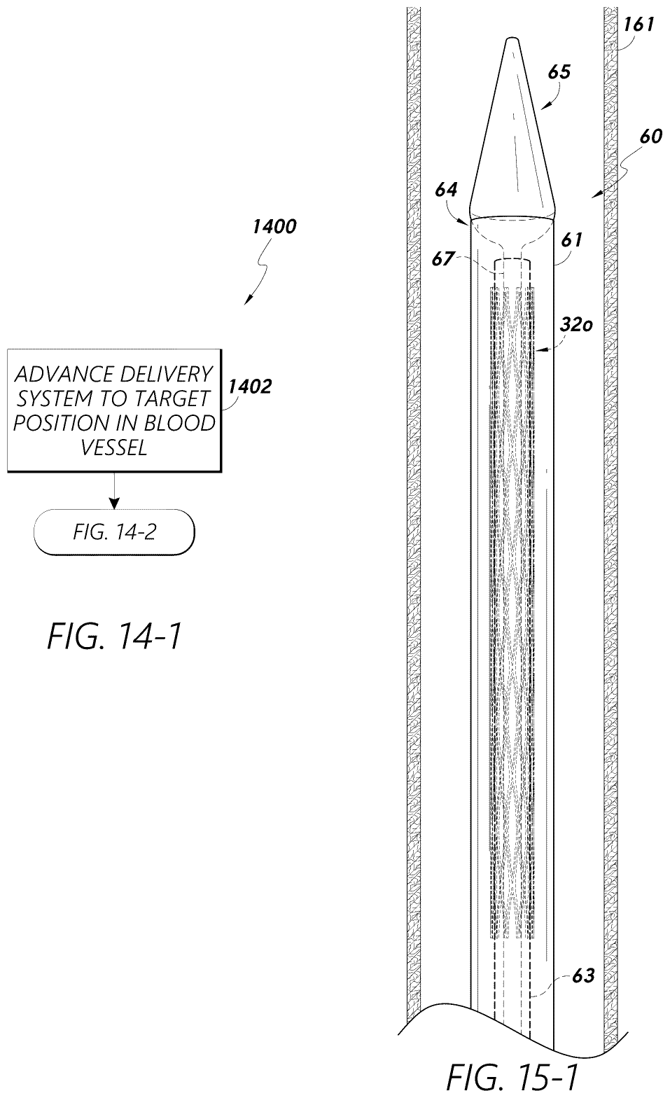

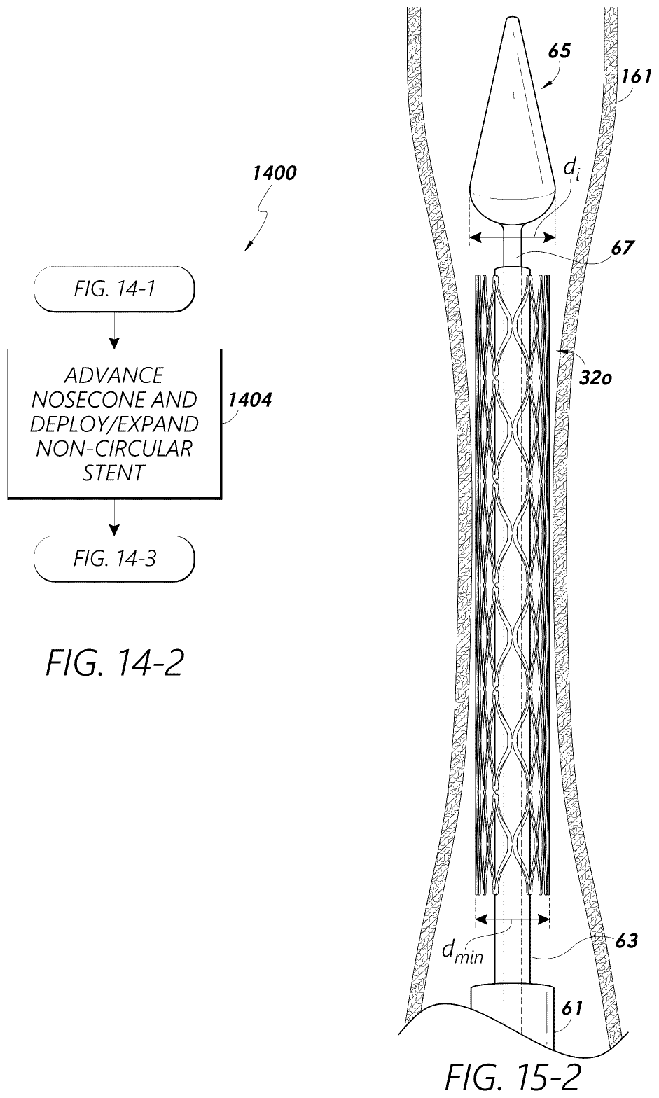

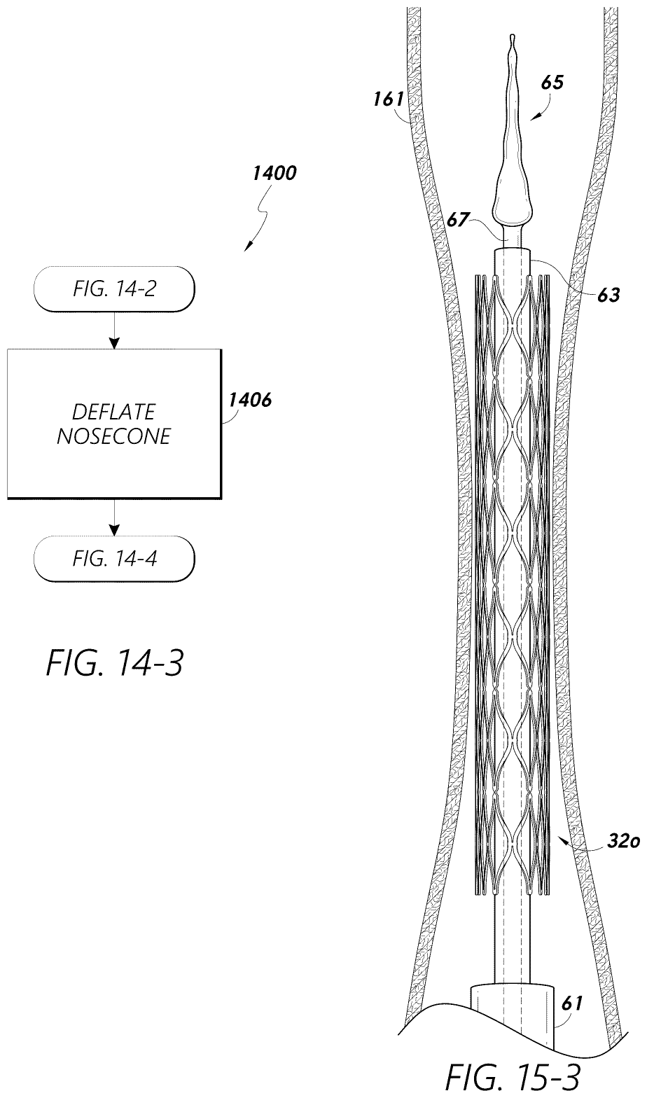

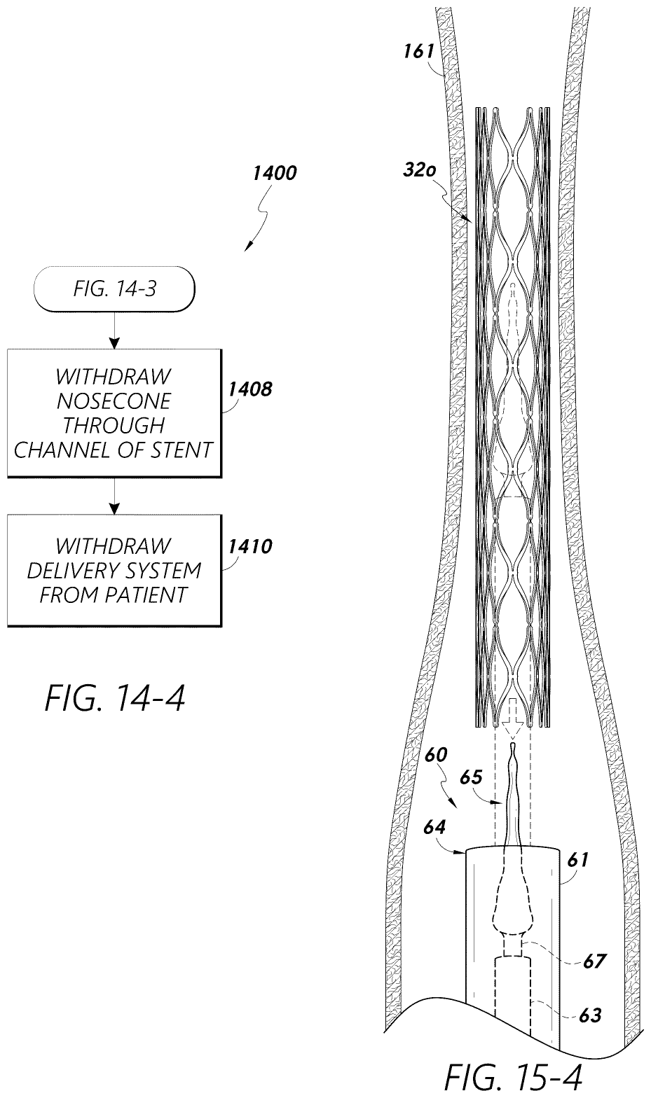

FIGS. 14-1, 14-2, 14-3, and 14-4 illustrate a flow diagram for a process 1400 for deploying an implant device 32o using a delivery system 60 with an inflatable nose cone 65 in accordance with one or more examples. FIGS. 15-1, 15-2, 15-3, and 15-4 provide images of the delivery system 60 and certain anatomy corresponding to operations of the process 1400 of FIGS. 14-1, 14-2, 14-3, and 14-4 according to one or more examples.

At block 1402, the process 1400 involves advancing a delivery system 60 that comprises an inflatable or otherwise modifiable nose cone 45 to a target position in a blood vessel 161, such as the aorta (e.g., abdominal or thoracic aorta), the delivery system 60 containing an implant device 32o that the delivery system 60 is configured to deploy. FIG. 15-1 shows the example delivery system 60 positioned in the blood vessel 161, wherein the delivery system 60 has disposed in a distal portion/capsule thereof the implant device 32o in a crimped/compressed delivery configuration. For example, the implant device may comprise an oval/non-circular stent comprising a wireframe that is configured to be radially compressed for delivery and held within the sheath 61 of the delivery system 60. The delivery system 60 includes the inflatable nose cone 65 disposed at a distal end 64 of the outer capsule/sheath 61, such that the nose cone 65, as inflated, provides an atraumatic leading end/edge of the delivery system 60 and prevents edges of the distal end 64 of the outer sheath 61 from abrading against the patient anatomy.

At block 1404, the process 1400 involves advancing the nose cone 65 relative to the distal end 64 of the sheath 61 to deploy/expand the non-circular stent/implant 32o. For example, as shown in FIG. 15-2, the nose cone 65 may be coupled to an adapter or other shaft component 63 on which the implant 32o is disposed during delivery. For example, the implant-holding adapter/shaft 63 may have the fluid lumen 67 used to inflate and/or deflate the nose cone 65 running therethrough, such as along as central axis of the shaft 63. As shown, the shaft 63 may be advanced distally relative to the sheath 61, thereby advancing the nose cone 65 and implant device 32o distally to unsheathe the implant 32o to allow for expansion thereof, which may be implemented using shape-memory/superelasticity characteristics of the device/frame 32o and/or through the use of a balloon expansion component (not shown).

When deploying the implant 32o, the nose cone 65 is positioned distal of the implant 32o, whereas the distal end 64 of the delivery sheath/capsule is positioned proximal of the implant 32o. In order to retrieve/withdraw the nose cone 65 back toward/into the delivery system 60/61 to thereby facilitate removal of the delivery system 60 from the patient anatomy while maintaining the implant device 32o in-place, it may be necessary to pass the nose cone 65 through the inner diameter of the implant device/stent 32o. However, where the implant device 32o has a minor diameter dₘᵢₙ that is less than the diameter dᵢ of the inflated nose cone 65, it may be necessary or desirable to deflate the nose cone 65 to thereby reduce a radial profile thereof to allow for the nose cone 65 to pass through the implant device 32o within the minor-axis diameter dₘᵢₙ thereof.

At block 1406, the process 1400 involves deflating the nose cone 65. For example, fluid may be aspirated from the nose cone 65 through the lumen 67. In some implementations, fluid may be drawn into a reservoir outside of the patient's body, or a reservoir disposed within the shaft/sheath 61 of the delivery system somewhere along a length thereof. The fluid aspiration may result in the deflation of the nose cone 65 and associated radial contraction thereof. FIG. 15-3 shows the nose cone 65 in the deflated state after fluid has been removed, wherein the deflated nose cone 65 has a reduced diameter that is sufficiently small to pass through the channel of the implant device 32o.

At block 1408, the process 1400 involves withdrawing/passing the deflated nose cone 65 through the inner diameter of the stent/implant 32o. Such withdrawal of the nose cone may involve proximally pulling the nose cone shaft 63, which may comprise the inflation/deflation lumen 67, relative to the distal end 64 of the sheath 61, such that the nose cone 61 approximates the distal end 64 of the sheath 61, or the sheath 61 and nose cone 65 may be proximally withdrawn in tandem, such that the nose cone 65 remains a distance distally from the end of the sheath 61. FIG. 15-4 shows the deflated nose cone 65 being pulled back towards/into the sheath 61, which may facilitate removal of the delivery system 60 with reduced risk of components of the delivery system 60, such as nose cone 65, being caught in the patient anatomy as the delivery system is withdrawn. Alternatively, the deflated nose cone 65 may remain distal to the end 64 of the sheath 61 during removal of the delivery system. At block 1410, the process 1400 involves withdrawing the delivery system 60 from the patient, thereby retaining the implant 32o in place indefinitely as a flow-control or other treatment means/mechanism.

Nose Cones with Proximal Taper Features

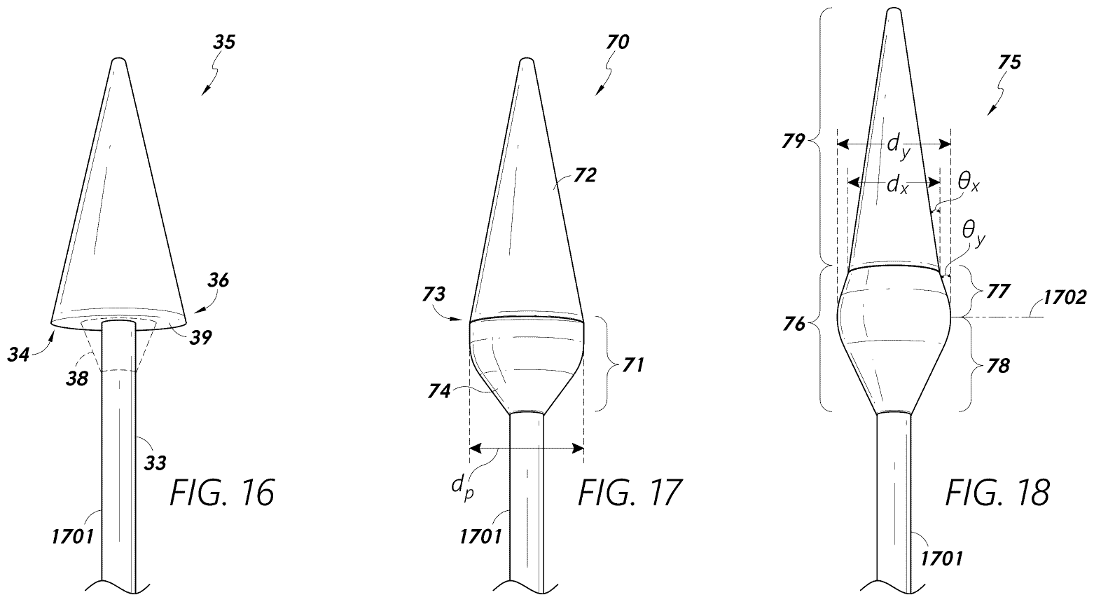

FIG. 16 shows a nose cone 35 having a proximal step/lip 34 in accordance with one or more examples. The proximal step feature 34, which may present a proximal surface 39 of the nose cone base 36 that is orthogonal to the axis of the nose cone 35. The proximally-facing surface 39 may span between the outer diameter of the nose cone base 36 and an outer diameter of a nose cone shaft 33 and/or shaft-transition component 38, which may have an outer diameter that is less than the outer diameter of the nose cone base 36.

The step 34 may serve to facilitate alignment of the nose cone 35 with the distal end of a sheath/capsule associated with the nose cone and associated delivery system. For example, the nose cone 35 may be configured such that the step 34 is configured to contact the distally-facing edge/end of the sheath, whereas the shaft 33 and/or transition portion 38 may be configured to nest within the distal portion of the sheath, thereby holding the nose cone 35 in axial position/alignment with the sheath. The transition portion 38, where implemented, may include a tapered surface, which may facilitate smooth transition into the sheath when the transition portion 38 contacts the distal end of the sheath. The transition portion 38 may taper to the nose cone shaft 33, which may have a diameter less than the outer diameter of the nose cone base 36 and/or the outer diameter of the transition portion 38 where the transition portion 38 contacts the nose cone lip/surface 39.

While the presence of the step/lip edge 34 of the proximal base 36 of the nose cone 35 can help with delivery sheath/capsule alignment, such exposed edge 34 can impede smooth retrieval of the nose cone 35 through a deployed implant device (e.g., inner diameter/channel of a deployed stent) due to the edge/step 34 presenting a surface 39 that can be prone to getting stuck/caught on implant features, such as stent struts or the like. Furthermore, the step/edge 34 of the nose cone 35 can cause abrasions on the patient anatomical tissue when being proximally pulled due to the angle of the edge thereof that can scrape the tissue. Therefore, it can be desirable to implement nose cones for delivery systems, wherein the nose cones comprise atraumatic tapered proximal features associated with the base of the nose cone that eliminate the presence of relatively sharp steps/edges as described above.

FIG. 17 shows a nose cone 70 having a tapered proximal end 71 in accordance with one or more examples. The nose cone 70 includes a proximal taper feature 71, which may be an integrated form with the body 72 of the nose cone 70, or may be a separate component formed or attached to the proximal base 73 of the nose cone 70. For example, the tapered feature 71 may serve as a transitional structure between the conical portion 72 of the nose cone 70 and the nose cone shaft 1701 that couples to the nose cone 70. The tapered feature 71 provides a graduated transition from the wider diameter of the proximal base 73 of the nose cone 70 to the relatively smaller diameter of the nose cone shaft 1701. Furthermore, the taper of the nose cone tapered structure 71 can help to open/expand a stent or other implant device through which it is withdrawn for the purpose of allowing passage of the nose cone 70 therethrough. That is, the tapered surface 74 of the proximal tapered form 71 can urge-apart certain implant portions/walls to allow for the nose cone 70 to be introduced into the channel/lumen defined by the implant and passed therethrough.

The proximally-tapered component 71 may advantageously have an outer diameter de that is equal to or greater than the outer diameter of the base 73 of the nose cone 70 and/or greater or equal to the greatest outer diameter of the nose cone 70. The tapered component 71 may have a tapered portion 74, wherein the taper thereof may extend all the way to the proximal base 73 of the nose cone 70, or the taper feature 74 may have a length/portion that is not tapered, wherein the taper portion 74 of the tapered feature 71 is associated with a proximal portion of the tapered feature 71, as shown in FIG. 17.

The tapered feature 71 may comprise metal or other substantially hard material, which may provide certain benefits. For example, such materials may have a relatively low friction coefficient with respect to contact with a metal stent frame or other implant device/component, thereby facilitating the gliding/passage of the tapered feature 71, and therefore the nose cone 70, through the channel of the implant. Furthermore, metals or other sufficiently hard materials may be hard enough to prevent fragments/chunks from being scraped and/or chipped off of the tapered feature 71 when the nose cone tapered feature 71 scrapes against and/or contacts metal struts of an implant device. For example, where soft material is used for the tapered feature 71, scraping of implant features against such component may cause the implant to get stuck/embedded into the tapered feature and/or dislodge and/or otherwise scrape-off particles/pieces of the tapered feature 71, which can enter the bloodstream and present risks of physiological injury.

The taper portion 74 of the tapered feature 71 may have any suitable or desirable angle relative to the axis of the nose cone. In some implementations, the taper 74 extends the entire length of the tapered feature 71. The tapered feature 71 may reduce to a diameter at the proximal end thereof that matches the diameter of the nose cone shaft 1701 extending proximally therefrom. The tapered feature 71 advantageously covers the proximal step of the nose cone base 73, thereby preventing exposure of any edges associated therewith. In some implementations, the tapered feature 71 radially covers a substantial/majority portion of the proximal step of the nose cone 70, but some reduced/minor radial edge of the step remains exposed, wherein such edge facilitates delivery system sheath alignment, while reducing the impact of the step of the nose cone 70 with regard to propensity towards becoming caught or stuck during withdrawal.

FIG. 18 shows a nose cone 75 having transitional distal and proximal tapering in accordance with one or more examples. The nose cone 75 comprises a tapered feature 76, which may have both distally-facing 77 and proximally-facing 78 tapered surfaces/portions, as shown. The distally-facing tapered surface 77, for example, may provide transitional tapering with the taper of the nose cone body/tip 79, such that the taper angle θ_{y} of the distally-tapered portion 77 of the feature 76 is greater than the taper angle θₓ of the nose cone body.

The tapered feature 76, due to the distal taper portion 77 thereof, can expand to a diameter d_{y} that is wider than the diameter dₓ of the nose cone body, wherein at the widest diameter/portion 1702 of the tapered feature 76, the tapered feature 76 transitions to the proximally-facing taper 78, which provides a graduated transition from the expanded diameter d_{y} of the tapered feature 76 down to a diameter that is closer matched to the diameter of the nose cone shaft 1701.

The larger diameter d_{y} of the tapered feature 76 may match and/or exceed the diameter of the delivery system outer sheath/capsule at a distal end thereof, thereby providing an atraumatic leading end/surface for advancing the delivery system when the nose cone 75 is placed against the distal end of the sheath/capsule with at least a portion of the proximal tapered portion 78 extending into the sheath/capsule.

### Flexible Nose Cone Cover Features

As described in detail above, withdrawal of a nose cone back through a deployed implant device may be necessary or desirable in some implant procedures. However, where a nose cone has dimensions that are wider than dimension(s) of a channel/passage through a deployed implant device through which the nose cone must pass for retrieval, retrieval can be impeded or prevented due to contact interference between the nose cone and the implant device. Therefore, it can be desirable to utilize nose cones having relatively small/reduced diameter/profile (e.g., diameter/profile smaller than that of the delivery system sheath/capsule used with the nose cone) to facilitate retrieval of the nose cone through a deployed implant device. However, nose cones that have a base diameter that is less than the outer diameter of the delivery system sheath/capsule used to deliver the implant device may not provide sufficiently wide diameter to shield and guide the delivery system sheath/capsule when advancing through the patient anatomy (e.g., vasculature). In some implementations, examples of the present disclosure provide delivery systems with relatively narrow nose cones, wherein such nose cones and/or associated delivery systems/sheaths include flexible covers that cover and/or span the gap in diameter between the narrow nose cone and the delivery system sheath/capsule to thereby provide for atraumatic insertion of the delivery system while allowing for easy retrieval of the nose cone due to the relatively narrow diameter/profile thereof.

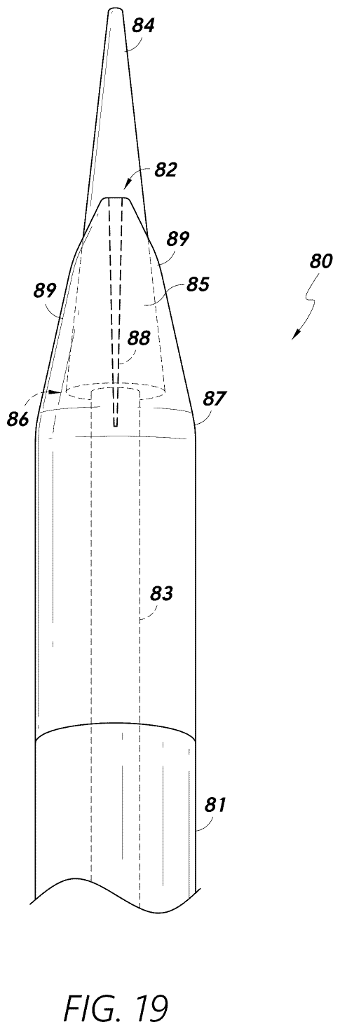

FIG. 19 shows a delivery system 80 including a narrow nose cone 85 and a flexible cover 87 in accordance with one or more examples. The flexible cover 87 may be coupled to and/or associated with the distal end/portion of a delivery system outer sheath/capsule 81. For example, the cover 87 may be attached to the distal end of the sheath 81 and/or integrated therewith as a unitary form/portion with the sheath 81. In some implementations, the cover 87 has an at least partially conical and/or tapered shape, wherein the nose cone 85 can be disposed within the delivery system 80 and/or cover 87 such that the distal tip 84 thereof can be passed through/out-of the cover 87 in a delivery configuration of the delivery system, as shown in FIG. 19.

In some implementations, the flexible cover 87 comprises one or more flaps 89, which may be separated by one or more longitudinal slits, cutouts, and/or gaps 88, which may be distributed circumferentially around at least a portion of the cover 87. The flaps 89 may be biased towards each other to form a tapered leading end/surface, which, in combination with the exposed portion 84 of the nose cone 85, provide for smooth advancement of the delivery system through the patient anatomy.

At least a portion of the nose cone 85 may be concealed/covered by the cover 87 during delivery for example, a proximal base/end 86 of the nose cone 85 may be maintained within the cover 87 during delivery, as shown. The natural tapering form/shape of the portion of the cover 87 that spans between the distal end of the sheath 81 and the distal end of the cover 87 can function as an extension of the nose cone 85 during delivery to the site of implantation. In some implementations, the flaps 89 and/or other portions of the cover 87 may be configured to be flexibly deflected radially outward, such that upon reaching the implantation site, the nose cone 85, as well as the nose cone shaft 83 and implant disposed thereon can be advanced through the cover 87, thereby opening-up the distal opening 82 of the cover 87 (e.g., urging open flaps 89 of the cover 87). Although flexible nose cone covers are described herein as having radially deflectable flaps, it should be understood that in some implementations, such covers may not have flaps, but rather may be configured to stretch to expand an opening thereof as a nose cone is pushed through the opening. For example, such covers may comprise a drape configured to collapse and stretch to provide smaller and larger openings therein.

The delivery system/assembly 80 effectively provides nose cone functionality through the operation of two distinct components, namely the nose cone 85 and the cover 87, which may be associated with the delivery system sheath 81. For example, with the nose cone 85 and cover 87 assembled together as shown in FIG. 19, the components can collectively provide the function of a nose cone with respect to advancement through the patient anatomy, whereas during implant deployment, the nose cone 85 and cover 87 can separate, such that the cover 87 is proximal of the implant device and nose cone 85 when the implant device is deployed, thereby allowing for the narrower nose cone 85 to be retrieved through the implant device.

The cover 87 may comprise any suitable or desirable at least partially flexible material, such as a flexible polymer, which may or may not have braided wire embedded therein. In some implementations, the flaps 89 of the cover 87 are not flexible, but rather are configured to radially deflect with respect to distal portions thereof using one or more hinge features or other mechanical features. In some implementations, the nose cone component 85 may be omitted, such that the sole nose cone feature of the delivery system is the cover.

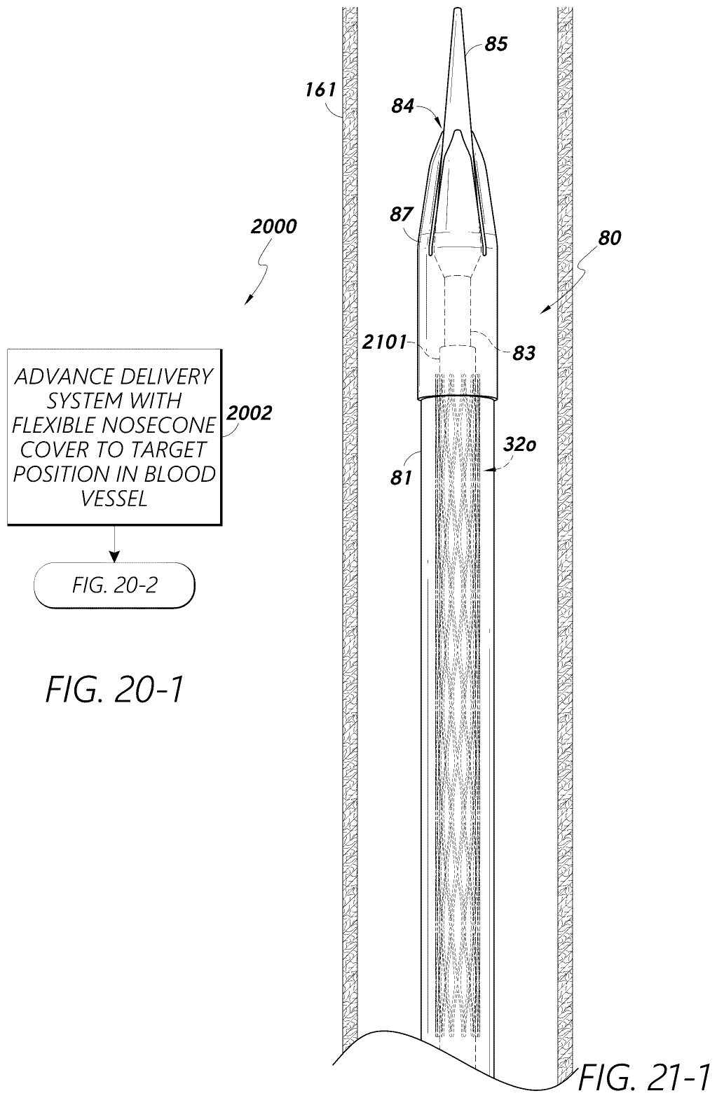

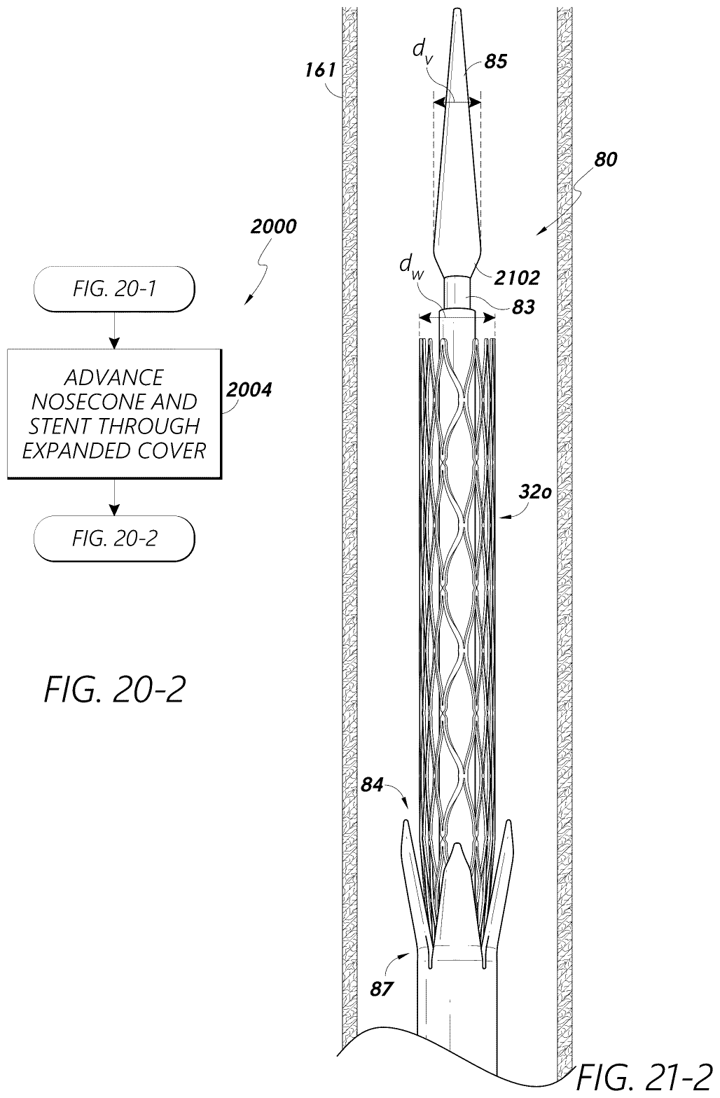

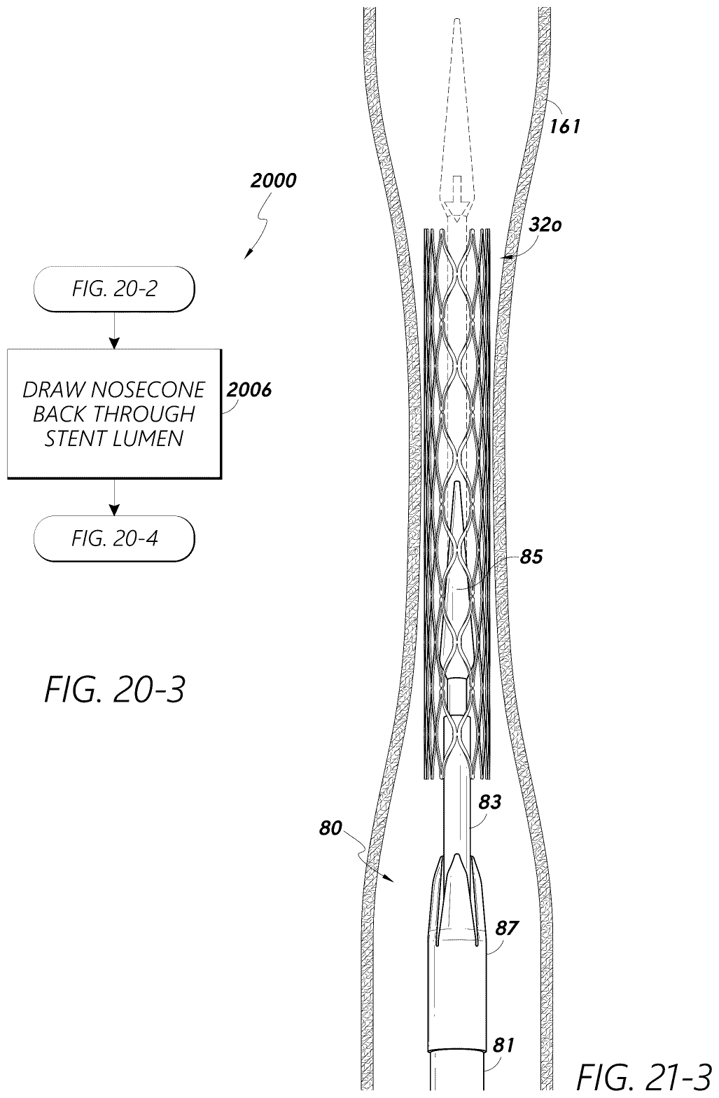

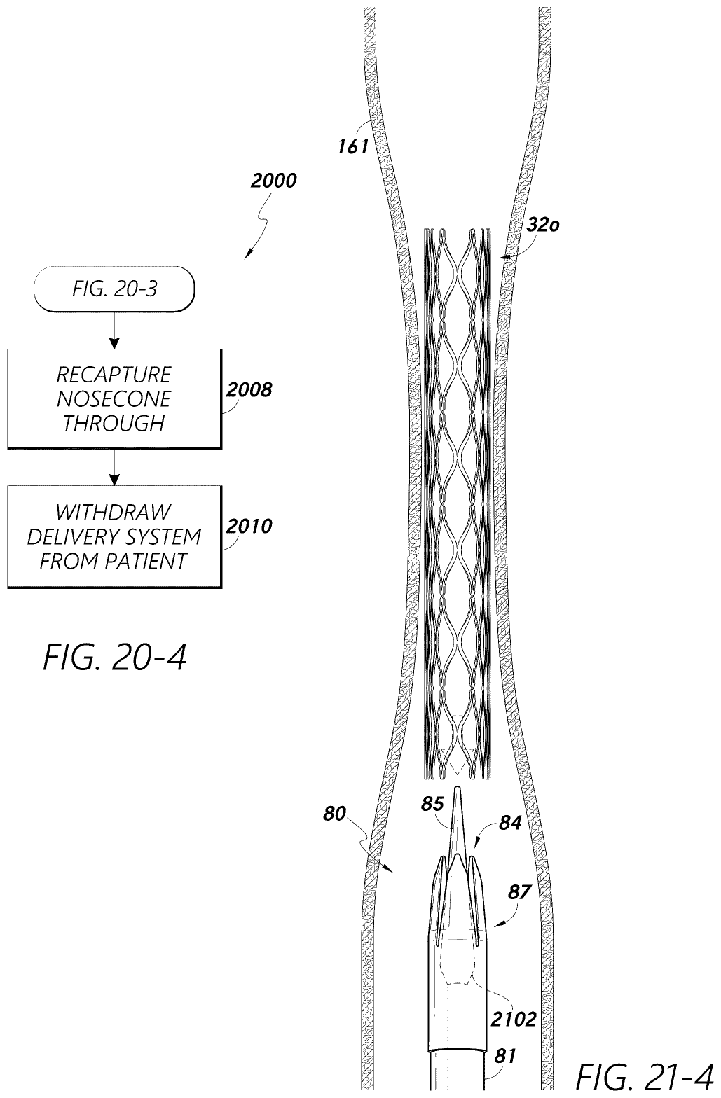

FIGS. 20-1, 20-2, 20-3, and 20-4 illustrate a flow diagram for a process 2000 for deploying an implant device 32o using a delivery system 80 with a flexible nose cone cover 87 in accordance with one or more examples. FIGS. 21-1, 21-2, 21-3, and 21-4 provide images of the delivery system 80 and certain anatomy corresponding to operations of the process 2000 of FIGS. 20-1, 20-2, 20-3, and 20-4 according to one or more examples.

At block 2002, the process 2000 involves advancing the delivery system 80 to a target position in a blood vessel 161, wherein the delivery system 80 comprises a flexible nose cone cover 87 at least partially covering a narrow nose cone 85, which projects from a distal opening 84 in the cover 87. FIG. 21-1 shows the delivery system 80 with the stent implant device 32o disposed in a sheath/capsule 81 thereof, wherein the implant device 32o is disposed on a nose cone shaft 83, which may include an adapter component 2101 configured to hold the implant 32o. The nose cone 85 may protrude from a distal opening/end 84 of the cover 87. The cover 87 is shown as being coupled to, or otherwise associated with, the delivery sheath/capsule 81.

At block 2004, the process 2000 may involve advancing the nose cone 85 and implant 32o through the distal opening 84 of the cover 87. For example, the nose cone 85 may be distally advanced relative to the sheath 81, thereby urging the distal opening 84 of the cover 87 apart to expand the opening 84 to permit passage therethrough of the nose cone 85, nose cone shaft 83, and implant device 32o. FIG. 21-2 shows an oval/non-circular stent 32o at least partially advanced through the cover 87 and expanded, with the nose cone 85 positioned distal of the stent 32o and cover 87 and the nose cone shaft 83 running through the stent 32o and passing through the opening 84 in the cover 87. Although advancement of the nose cone 85 is described, it should be understood that relative movement of the nose cone 85 and implant device 32o relative to the sheath 81 may be achieved by proximally retracting that sheath in addition to, or rather than, distally advancing the nose cone 85. When the nose cone 85 is advanced, the nose cone assembly, which includes the cover 87 and the nose cone 85, may become separated into two axially-offset components, as shown. When separated from the cover 87, the nose cone 85 advantageously has a maximum diameter dᵥ that is less than the diameter of the sheath 81 and/or cover 87, thereby providing a smaller-profile distal delivery system component that is more easily navigated through anatomy and the stent.

At block 2006, the process 2000 involves drawing the nose cone 85 proximally back through the lumen of the expanded stent implant 32o. FIG. 21-3 shows the nose cone 85 passing through the stent channel/lumen, wherein the nose cone 85 may ultimately be pulled to a position proximal of the implant device 32o, thereby permitting removal of the delivery system. The stent 32o may be an oval stent as described herein, wherein the image of FIG. 21-3 shows a minor-axis side view of the stent 32o showing the shorter diameter dimension thereof.

At block 2008, the process 2000 involves recapturing the nose cone 85 into the cover 87. In some implementations, the nose cone 85 may not be brought back into the cover 87 prior to withdrawal from the patient anatomy. FIG. 21-4 shows the nose cone 85 being drawn back into the cover 87. For example, the nose cone 85 may have a tapered proximal feature 2102 that facilitates expansion of the opening 84 of the cover to accommodate the nose cone 85. That is, the cover 87 may be biased to a closed configuration in which the flaps/opening 84 thereof are biased to a relatively small opening, wherein the tapered proximal feature 2102 of the nose cone 85 may serve to urge the opening 84 of the cover 87 open to receive the nose cone 85. At block 2010, the process 2000 involves withdrawing the delivery system 80 from the patient anatomy, thereby leaving the implant device in place for therapeutic purposes.

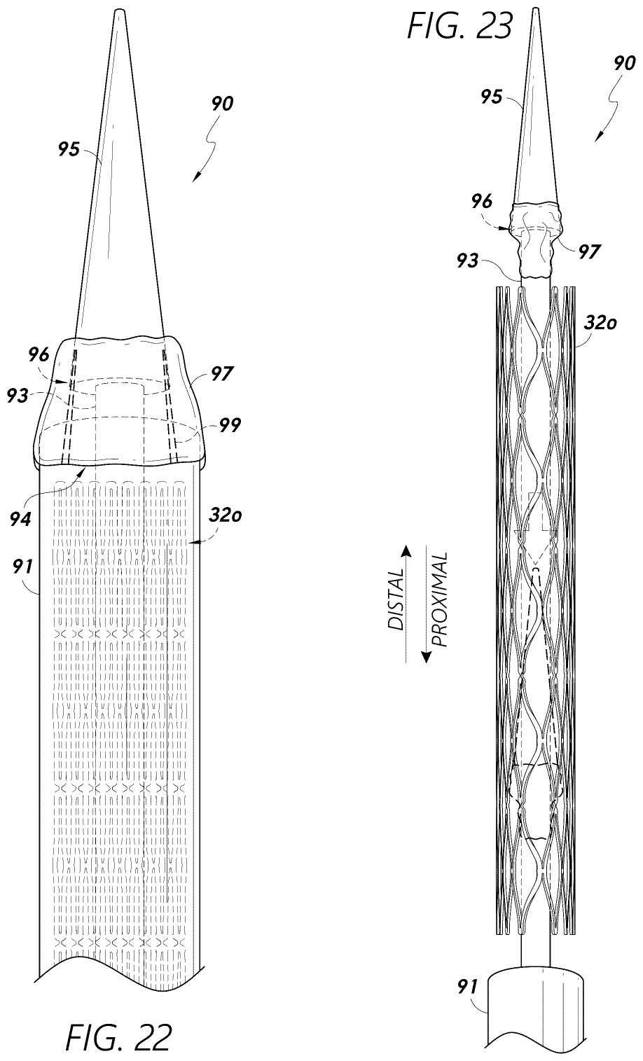

FIG. 22 shows a delivery system 90 including a nose cone 95 comprising a proximal flexible cover 97 in accordance with one or more examples. The delivery system 90 of FIG. 22 provides an alternative implementation of a nose cone cover, wherein the nose cone cover 97 is fixed to the nose cone component 95 rather than the sheath 91, as is described above. The opening 94 of the cover 97, which may or may not comprise slits 99, extends proximally from the proximal portion/end 96 of the nose cone 95, rather than distally as in the example described above. During delivery, the flaps/opening 94 of the flexible cover 97 can extend over the distal portion of the capsule/sheath 91, thereby covering a sheath 91 for delivery to prevent damage to the anatomy and/or to prevent the sheath from getting stuck on instrumentation or anatomy.

Upon reaching the implantation site, the nose cone 95 may be advanced relative to the capsule/sheath 91, such as by pushing the nose cone 95 forward distally or proximally pulling the sheath 91 back to unsheathe the nose cone shaft 93 and/or implant 32o. Separation of the nose cone 95 from the distal end of the sheath/capsule 91 allows the flaps/drape 97 to radially retract/compress/contract toward the nose cone shaft 93 and/or toward an axis of the nose cone 95 resulting in a narrower profile for the nose cone 95 that can more easily be retrieved through the deployed stent 32o. The cover 97 may be considered part of the nose cone 95. For example, the maximum diameter of the expanded cover 97 shown in FIG. 22 may be considered the maximum diameter of the nose cone 95.

FIG. 23 shows the delivery system 90 of FIG. 22 deploying the stent implant 32o in accordance with one or more examples. With the nose cone 95 displaced from the distal end of the sheath 91 for implant deployment, the cover 97 may collapse due to the biasing thereof towards a compressed/collapsed profile, thereby reducing the diameter of the nose cone 95. FIG. 23 further shows, in dashed line, the nose cone 95 being proximally pulled back through the deployed stent 32o with the collapsed cover 97 on the leading side of the retrieval.

The nose cone 95 shown in FIGS. 22 and 23 effectively provides a nose cone that is configured to transition from a wider nose cone configuration during transit/delivery to a narrow-profile, post-deployment nose cone for proximal passage through the minor-axis or other dimension of the deployed implant/stent 32o. The cover 97 can comprise one or more slits 99, or may have a drape form that does not include slits. For example, with respect to a drape configuration, such material of the drape 97 may be at least partially flexible/stretchy to accommodate stretching over the distal end of the sheath 91, wherein elasticity of the material causes the drape to collapse once removed/displaced from the distal end of the sheath 91.

Nose Cones with Collapsible Frames

For percutaneous/transcatheter procedures, as described in detail herein, such procedures may be implemented using delivery systems comprising catheter or other sheath/shaft devices having associated therewith a distal tapered tip, referred to herein as a nose cone. When therapy is provided using such delivery systems, which may involve the deployment of one or more implant devices, such as a stent or other device including an anchoring frame, at least a portion of the delivery system, including the distal nose cone, may need to be removed from the anatomy through a channel of the implant device, as described in detail herein. Descriptions herein of nose cone retrieval processes refer to such procedural steps. As described in detail above, during nose cone/device retrieval, the nose cone may become tangled or caught on portions of the anatomy and/or on edges/portions of an implant device deployed and intended to be left behind in the anatomy, Generally, as a nose cone may be positioned distally to the deployed implant, which may be disposed on a nose cone shaft or other component(s) associated with the delivery system sheath/catheter during delivery, recapture of the nose cone after implant deployment may require passage back through the implant.

With respect to procedures for deploying/implanting stents or other frame-based implant devices, such implant devices may form their expanded, deployed shape/configuration only after becoming disconnected from the delivery system (e.g., nose cone shaft/catheter). For example, aspects of the present disclosure relate to the deployment of shape-memory/superelastic metallic alloy implants, such as nitinol stents or frames. In some cases, the final implant shape when deployed may have a cross-sectional dimension that is or becomes smaller than the largest outer diameter of the nose cone and/or delivery system sheath/capsule. In such cases, it may be desirable to configure the nose cone in a manner as to allow the nose cone to assume a profile sufficiently narrow to pass back through the implant without disturbing the implant's resting location/position in the anatomy. Furthermore, other concerns associated with nose cone profile may likewise encourage lower-profile nose cone features. For example, nose cones having a diameter associated with at least a base thereof that matches or exceeds that of a delivery system sheath/capsule may present concerns with respect to damaging, by the nose cone, of relatively fragile anatomical features during nose cone/device retrieval, whereas such concerns may not be as germane during the delivery system insertion. For example, a tapered nose cone may have features associated with the proximal base thereof that are less atraumatic compared to the distal narrowed tip/end of the nose cone.

For at least the foregoing reasons, it may be desirable to provide nose cone components/features that provide the ability to actively collapse the nose cone before through-implant retrieval in a manner as to reduce a diametrical profile thereof, at least with respect to a base portion of the nose cone. As with some of the examples described above, aspects of the present disclosure relate to delivery system nose cone features that are configured to be collapsed to thereby facilitate passage through relatively narrow channels of deployed implant devices in connection with device/nose cone retrieval. Implant devices that may have relatively narrow diameters in one or more portions thereof can include oval/non-circular stents, as described in detail herein, or other stents or implant devices, such as hourglass-shaped stents/implants, or any other device that when deployed, a cross-sectional inner diameter thereof is smaller than the outer diameter of a nose cone and/or delivery system sheath.

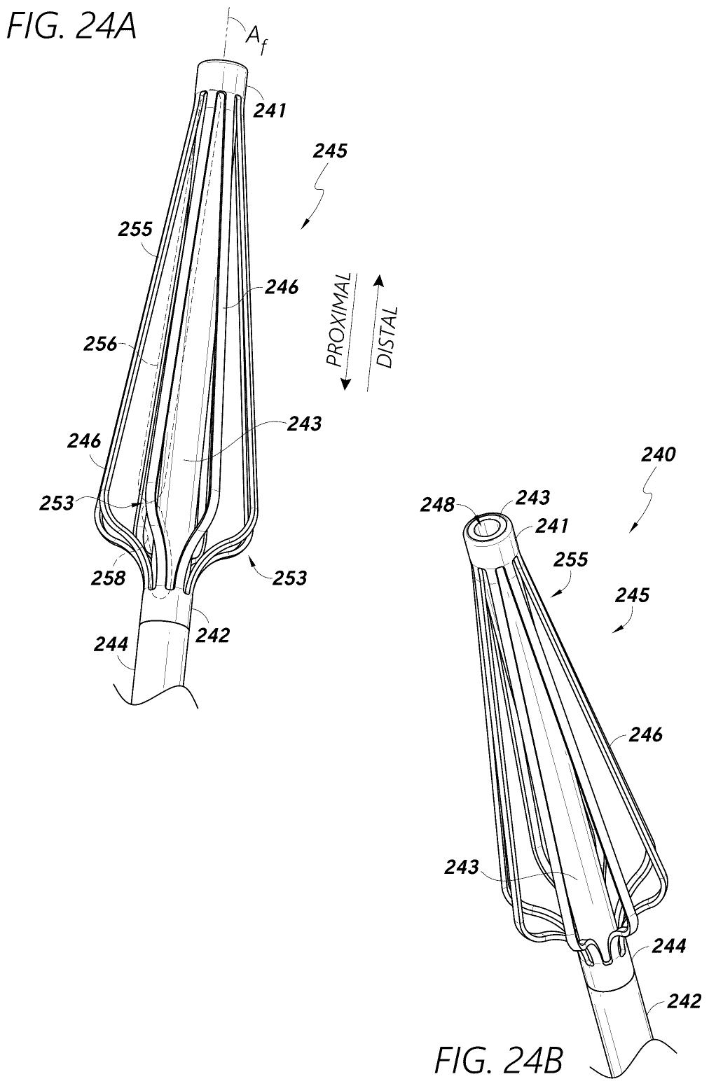

FIGS. 24A and 24B show perspective views of a collapsible nose cone 245 including a frame 255 with struts 246 in accordance with one or more examples. The nose cone 245 of FIGS. 24A and 24B may comprise a stent-type frame 255, which may comprise metal (e.g., shape-memory alloy) or other at least partially rigid material configured to assume expanded and compressed states in response to shape-memory characteristics thereof and/or manipulation of one or more portions or components thereof.

The nose cone frame 255 may comprise a plurality of longitudinal/axial struts 246 that span a length of the frame and are circumferentially offset from one another, as illustrated. In some implementations, the struts 246 of the frame 255 can be created by laser-cutting a tube of metal or other rigid material, such as a nitinol metal alloy tube. The struts 246 of the frame 255 may be mechanically deformed through, for example, fixturing or other process. After the struts 246 are formed in a radially deflected/expanded state, the frame 255 may be heat-set or otherwise shape-set to maintain the desired nose cone shape. For example, the struts 246 may be deflected radially outward such that they form a bend 253, wherein the struts 246 are angled radially inwardly towards the axis A_{f} of the frame 255 moving towards the distal end thereof. Therefore, the frame 255 may comprise distally-tapered strut segments 256, as well as proximally deflected and/or tapered segments 258 separated by bends 253, In some implementations, the frame 255 may be formed by welding a series of wires (e.g., nitinol wires) between proximal 244 and distal 241 rings/collars, wherein the wires/struts may be shape-set, such as through heat-setting.

The nose cone frame 255 may be collapsible by proximally pulling/actuating a proximal collar/structure 244 of the frame, which may cause elongation of the frame struts 246. For example, the distal ends of the struts 246 may be coupled to a distal collar/structure 241 that is fixed relative to a structure, such as an internal guide wire lumen/shaft 243, which forms a channel/lumen 248 through which a guidewire may pass. Therefore, when the proximal collar is proximally drawn relative to the guidewire lumen/shaft 243, such actuation may increase the distance between the proximal 244 and distal 241 collars/structures, thereby causing radially-inward deflection of the bends 253 of the struts 246, and the struts 246 themselves, to a more elongated/straightened configuration, thereby reducing the diameter of the nose cone frame 255, such as at the area of the bends 253 in the struts 246. Therefore, elongation of the frame 255 may in turn cause reduction in profile of the frame.

The internal shaft 243 may serve as a guidewire lumen or may have another function and/or structure. The shaft 243 provides an anchoring structure/mechanism to allow for fixing of the distal end 241 of the frame 255 to allow for manipulation of the distance between the distal 241 and proximal 244 ends/collars of the frame 255. The distal end of the frame 255, such as the collar/frame 241 or other structure, may be welded or otherwise attached, adhered, or secured/fixed to the inner shaft 243, such as to a distal end thereof.

The proximal portion/collar 244 of the frame 255 may be coupled to and/or integrated with a shaft 242 that is slidingly disposed over the inner shaft 243, such that when the outer shaft 242 is proximally slid/translated relative to the inner shaft 243, the frame 255 is elongated and reduces in profile. The outer shaft 242 may comprise any suitable or desirable material, such as nitinol metal alloy, stainless steel, plastic, or the like. It may be desirable for the outer shaft 242 to have axial rigidity to provide for efficient translation of axial movement thereof to the proximal collar 244 of the frame 255. As proximally pulling the proximal collar/structure 244 of the frame 255 causes elongation of the frame 255 and struts 246, thereby reducing the profile of the frame 255, distal pushing of the proximal collar/structure 244 and/or otherwise shortening the distance between the proximal 244 and distal 241 ends of the frame 255 may cause shortening of the frame 255 and radially-outward expansion of the struts 246. It may be desirable for the inner shaft 243 and/or the outer shaft 242 to comprise a common material with that of the frame 255 in order to facilitate welding of such components to one another in a manner as to provide desirable structural integrity.

In some implementations, one or both of the inner shaft 243 and the outer shaft 242 may be cut along the length thereof, such as through laser cutting, in a manner as to provide bending flexibility along a length thereof for bending through tortuous anatomy, such as the vasculature of a patient. That is, it may be desirable to implement the inner shaft 243 and/or outer shaft 242 to have flexibility to allow for the bending thereof around curves and/or other anatomical features. In some implementations, the inner 243 and/or outer 242 shafts comprise flexible polymer. In some implementations, distal portions of the inner 243 and/or outer 242 shafts comprise rigid metal or other rigid material, whereas more-proximal portions thereof may comprise flexible polymer or other flexible material.

The proximal collar/structure 244 of the frame 255 and/or the outer shaft 242 are advantageously slidable over the inner shaft 243. Although shown as two separate components, it should be understood that the proximal collar/structure 244 of the frame 255 may be a single unitary form with the outer shaft 242. Furthermore, while the outer actuator shaft/structure 242 is shown as a tubular shaft, it should be understood that such future may be/comprise a rod, wire, or other elongate feature not forming a complete tube around the inner shaft 243.

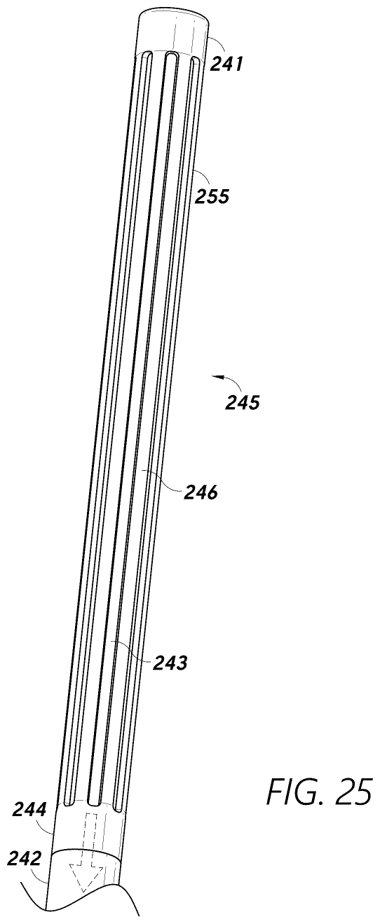

FIG. 25 shows the nose cone frame 255 of FIGS. 24A and 24B in a collapsed state in according with one or more examples. As referenced above, in order to collapse the nose cone frame 255, the proximal outer tube/shaft 242, which may be welded to, or otherwise coupled to and/or integrated with, the nose cone frame 255, may be actuated in some manner to move proximally relative to the inner shaft 243, which is coupled to the distal end 241 of the nose cone frame 255. Such lengthening of the frame 255 may be achieved by proximally pulling the outer shaft 242 and/or by distally pushing/advancing the inner shaft 243, thereby producing axial displacement between the inner 243 and outer 242 shafts. The proximal collar/structure 244 and/or the outer shaft 242 may be proximally pulled in any suitable or desirable manner, such as through the actuation of a lead screw or other mechanical mechanism associated with a more proximal portion of the delivery system. For example, such actuator(s) may be associated with a handle of the delivery system that is configured to be disposed outside of the patient's body when the nose cone 245 is disposed within the patient anatomy. Actuation of the nose cone frame 255 may be in a continuous manner, wherein many different states of expansion and/or elongation of the frame 255 are possible, or the frame 255 may be inclined to assume one of two positions corresponding, respectively, to the expanded/shortened configuration shown in FIGS. 24A and 24B and the compressed/elongated configuration shown in FIG. 25.

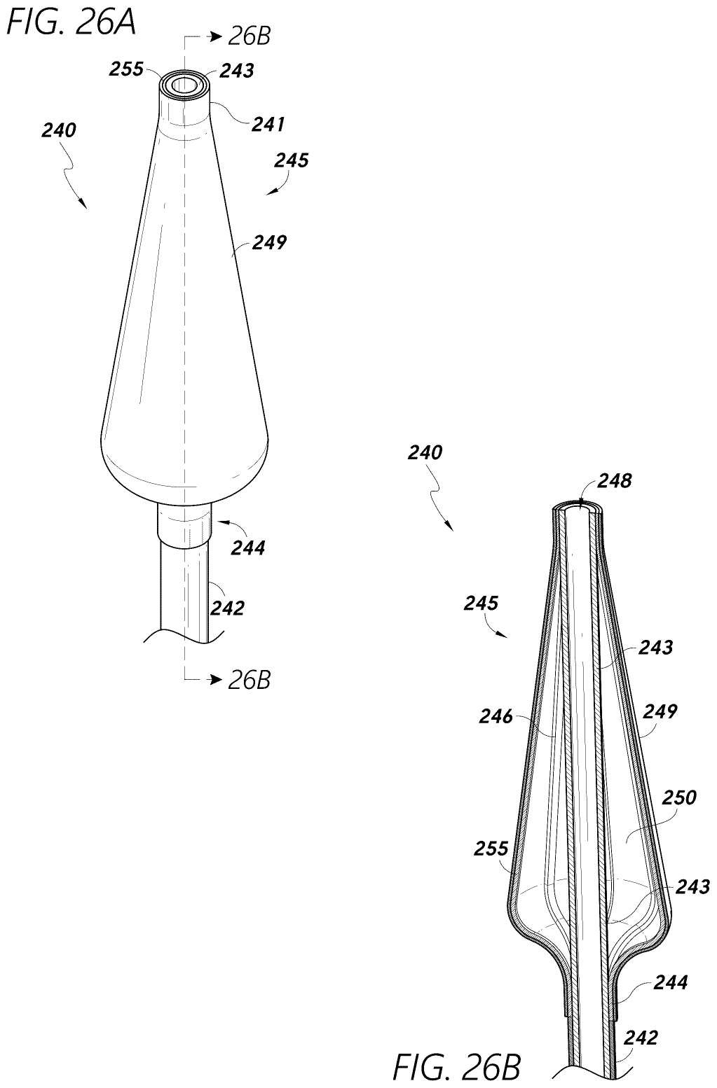

FIGS. 26A and 26B show perspective views of a collapsible nose cone 245 including a frame 255 and cover 249 in accordance with one or more examples. Although not shown in FIGS. 24A, 24B, and 25 for visual clarity, it should be understood that nose cone frames disclosed herein may have cover features associated therewith. For example, as shown in FIGS. 26A and 26B, a fluid-type covering 249, which may comprise elastic polymer such as silicone, latex, or urethane, may cover at least a portion of the outer diameter of the frame 255. The cover 249 may advantageously comprise stretchable and/or elastic material to allow for the polymer to stretch to assume the expanded nose cone shape and contract to some degree during compression to thereby reduce the profile thereof in the collapsed configuration of the nose cone 245. In some implementations, the cover 249 is inelastic, wherein in the compressed state thereof, the cover may be inclined to wrinkle and/or fold to reduce the diameter thereof. In some implementations, pulling of the proximal portion 244 of the nose cone to contract the nose cone frame 255 may likewise elongate the cover 249 and reduce the profile thereof, wherein the cover 249 may be coupled to the frame 255 struts 246 and/or otherwise assume a position that lines the outer diameter of the struts 246.

The cover 249 (e.g., polymer cover) can be attached to one or more portions of the nose cone frame 255 through, for example, a heat-bonding process, such as reflow, or can be mechanically locked to the stent frame 255 with a ring feature or other fastener. For example, a fastening ‘O’-ring may be placed outside of the cover 249 at the distal and/or proximal collars/ends 241, 244 of the frame 255 to couple to the frame 255.

As shown in FIG. 26B, with the cover 249 disposed over the nose cone frame 255, a void 250 may be present between the cover 249 and struts 246 and the inner shaft 243. It may be necessary or desirable to fill such void 250 in order to prevent radial collapsing of the nose cone frame struts 246. In some implementations, the void 250 within the nose cone 245 may be filled with saline solution, carbon dioxide gas, or other liquid or gas, which may be injected into the internal space 250 of the nose cone 245 to expand the nose cone 245. Furthermore, the delivery system 240 may provide a means/mechanism for extracting/aspirating the fluid from the nose cone 245 to allow for collapsing thereof. Use of saline solution or other biocompatible liquid or filling in the nose cone 245 may be preferable to air or other gas, which may create issues with respect to venting of the nose cone 245 during collapsing thereof. In some implementations, a fluid lumen is provided in the delivery system 240 that traverses the space between the nose cone 245 and a more-proximal fluid reservoir, which may be disposed in a handle, or other area outside the patient's body during delivery, or the reservoir may be disposed in the delivery system shaft (243 and/or 242) in some position along the length thereof. The fluid lumen may be utilized to relieve the fluid from the nose cone 245 when collapsing the nose cone 245. For example, fluid may be aspirated from the nose cone 245 and/or fluid may be injected into the nose cone 245 to compress and expand the nose cone 245, respectively. In some implementations, the fluid lumen may be disposed between the inner 243 and outer 242 shafts/structures.

In some implementations, the space 250 between the cover 249 and the inner shaft 243 may be vacuum-filled. In such implementations, the frame struts 246 may necessarily be sufficiently strong to hold the cover 249 in the expanded nose cone shape without collapsing. Vacuum-filling of the nose cone 245 may be desirable to avoid complications that may be associated with rupture of the nose cone cover 249 in cases where fluid is filled therein.

The cover 249 may advantageously provide an atraumatic surface for the nose cone 245 that prevents the nose cone frame struts 246 from abrading and/or catching on anatomy and/or other structures. That is, the cover 249 may provide an atraumatic leading edge for the nose cone 245 and delivery system 240 to guide the delivery system 240 through the patient anatomy. The cover 249 may prevent the struts 246 from becoming caught or snagged on blood vessel walls and/or calcification formed thereon. Furthermore, the cover 249 may prevent fluid from accessing the delivery system sheath/capsule during delivery between the struts 246 and/or other gaps in the nose cone frame 255.

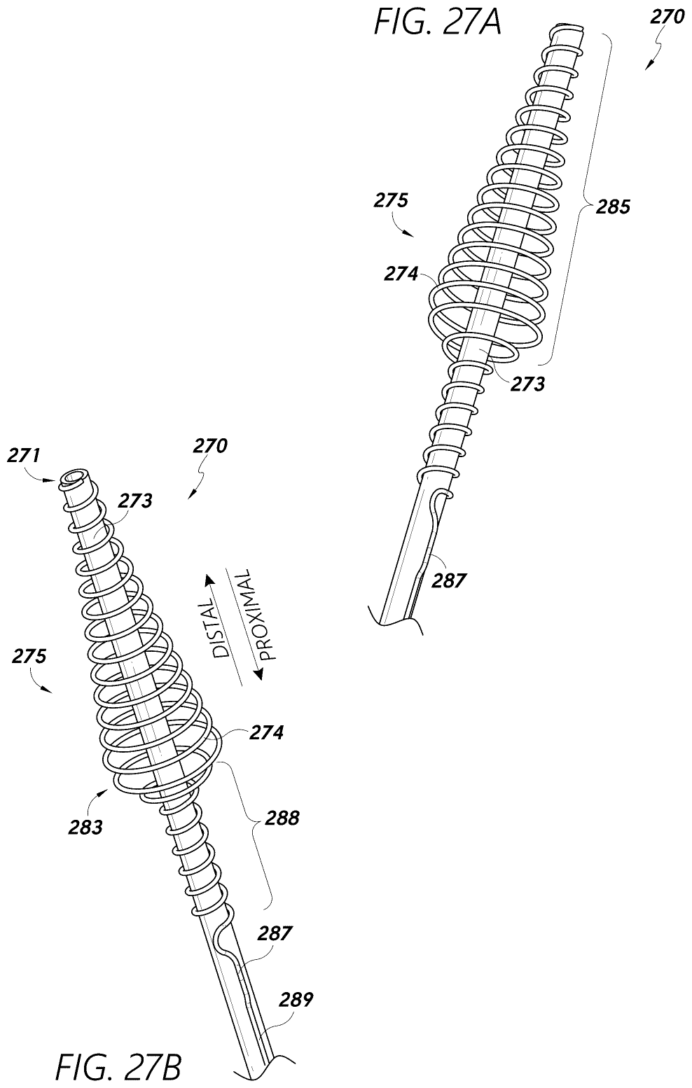

FIGS. 27A and 27B show perspective views of a collapsible nose cone 275 including a coil frame 285 in accordance with one or more examples. The nose cone frame 285 provides a different design from that shown in FIGS. 24A and 24B in that the frame 285 may comprise a wire 287 winding in a helical manner about an inner shaft 273, as opposed to longitudinal struts. The wire coil 287 may be formed of shape-memory metal alloy (e.g., nitinol) or other material configured to hold a tapered shape as shown in FIGS. 27A and 27B.

The expanded shape of the wire form coil 285 may be formed by winding/placing the wire in multiple axially-offset coils/winds 274 around a cone-shaped mandrel or other fixture and setting the shape thereof, such as through heat-setting or other process. The shape of the wire form coil/frame 285 in the expanded configuration shown in FIGS. 27A and 27B may be implemented by forming the coils 274 in coils of expanding diameter moving from the distal end 271 of the frame 285 to an apex/base 283 of the frame 285, after which the coils 274 may, moving in the proximal direction, reduce in diameter until wound relatively closely/tightly around the shaft.

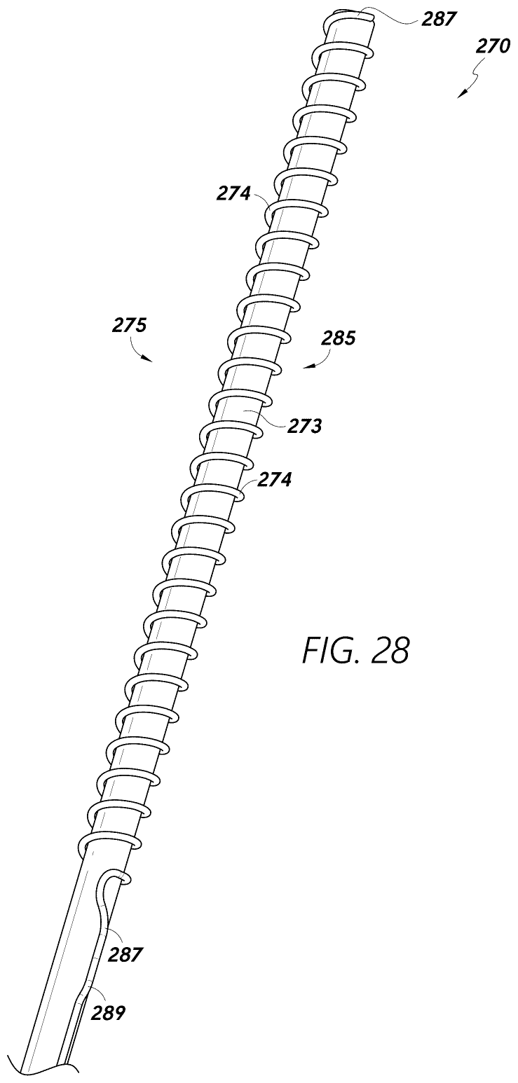

FIG. 28 shows the nose cone frame 285 of FIGS. 27A and 27B in a collapsed state in according with one or more examples. Collapsing of the coiled nose cone frame 285 may be implemented by proximally pulling on one or more portions of the coil wire 287. For example, the distal portion 271 of the coil 287 may be fixed/secured to the inner shaft 273, whereas the proximal winds 288 of the coil may be permitted to slide axially along the inner shaft 273. Therefore, pulling proximally on the proximal coils 288 and/or distally advancing the inner shaft 273 relative to the proximal coils 288 may cause the expanded coils of the frame 285 to collapse.

Actuation/pulling proximally on the wire 287 may be implemented using any suitable or desirable mechanism. For example, the wire 287 may be coupled to a lead screw or other actuator associated with the proximal portion of the delivery system 270 (e.g., handle), such that when the lead screw or other actuator is actuated, tension in the wire 287 causes the wire 287 to be pulled proximally and collapse-in the nose cone portion 285 thereof. Although pulling on the wire 287 is described as a mechanism for collapsing the coil-form nose cone frame 285, in some implementations, winding of the wire coils 274 may cause the coils to reduce a diameter and collapse around the inner shaft 273. The proximal portion 289 of the wire 287 may extend along the entire length of the delivery system shaft to a proximal actuator mechanism that may be manipulable by a surgeon/technician either mechanically or electronically (e.g. using an electric motor) to cause the frame 285 to collapse and/or expand.

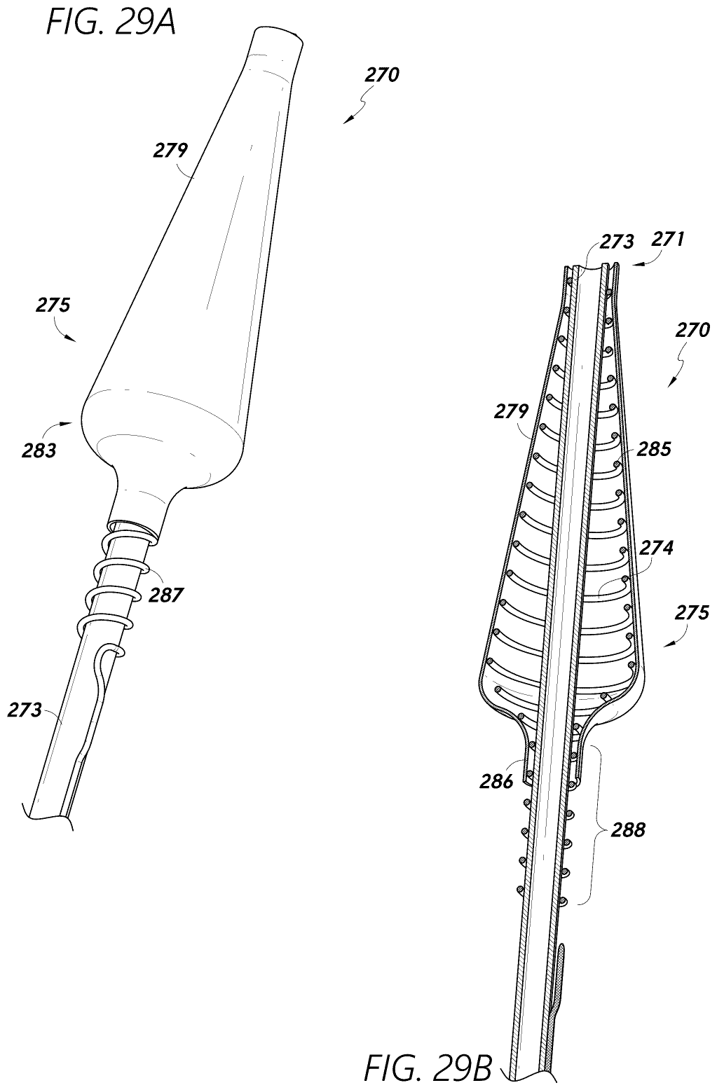

FIGS. 29A and 29B show perspective views of the collapsible nose cone 275 with a cover 279 covering the coiled frame 285 in accordance with one or more examples. The cover 279 may have any of the features disclosed above in connection with FIGS. 26A and 26B. The cover 279 may be assembled over the coil wireframe 285 of the nose cone 275, wherein the cover 279 may stretch to form the nose cone shape and contract when the coils 274 of the nose cone 275 are collapsed.

In some implementations, collapsing the coil wireframe 285 can cause the proximal portion 288 of the coils to pull/pass out of the proximal end 286 of the cover 279. In some implementations, the secure hold of the proximal portion 286 of the cover 279 on the wire coils 288 may provide friction to maintain mechanical coupling between the proximal portion 286 of the cover 279 and the wire coils 288 when the wire coils 288 are pulled proximally, thereby pulling the proximal portion 286 of the cover 279 along with the wire coils 288. In some implementations, a collar or other coupling/interface may be attached/coupled over and/or between the proximal portion 286 of the cover 279 and the proximal wire coils 288 to hold the proximal portion 286 of the cover 279 to the wire coils 288. In some implementations, the wire 287 may puncture and/or pass through the proximal portion 286 of the cover 279 to fix the wire 287 to the cover 279 to facilitate collapsing of both the coil frame 285 and the cover 279 through proximal pulling of the wire 287. In some implementations, collapsing of the nose cone 275 is implemented by pulling on the proximal portion 286 of the cover 279, which may force the collapsing of the nose cone 275 by applying radially-inward force on the coils 274 and/or by pulling the wire coils 274 proximally therewith.

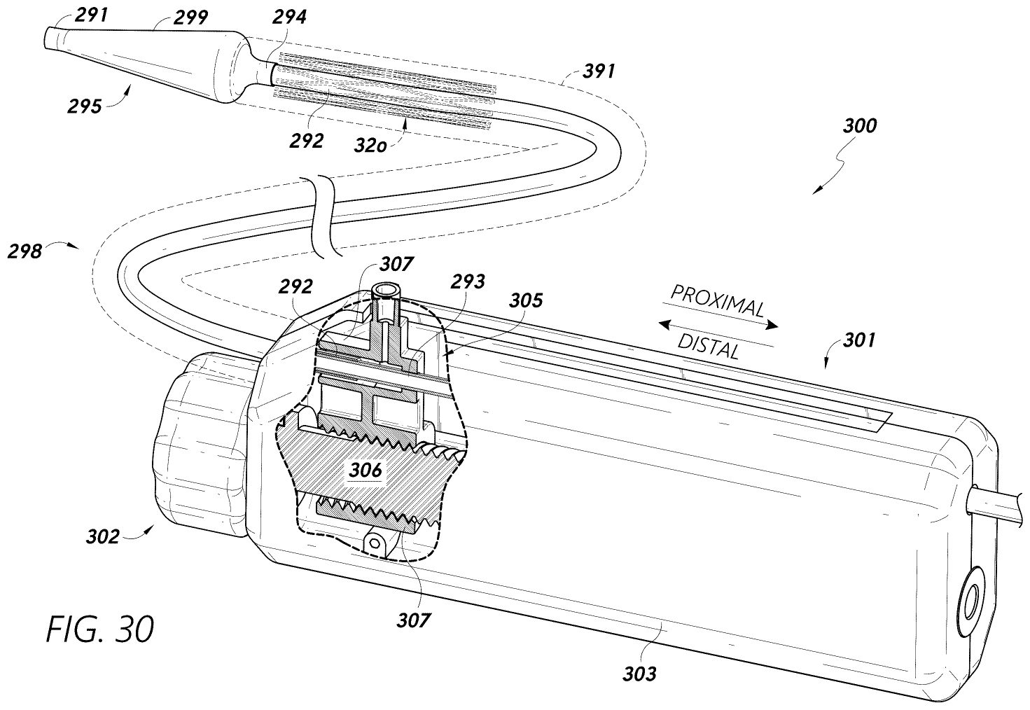

FIG. 30 shows a delivery system 300 comprising a collapsible nose cone 295 and a handle-based actuator 302 configured to collapse and/or expand the nose cone in accordance with one or more examples. The actuator 302 may comprise a knob-type feature and/or may be manually manipulable to cause radial expansion and/or retraction of the nose cone 295, which may be similar in one or more respects to either of the nose cones 245, 275 described above.

An outer frame-actuating shaft 293 may be provided comprising a sheath that extends over an inner nose cone shaft, which may form a guidewire lumen. The outer shaft 292 and inner shaft 293 may be disposed at least partially within an outer sheath 391, shown in dashed-line for visual clarity. The outer shaft 292 may have a proximal end that couples to a manually- or motor-driven actuator 305 of the handle 301, and may have a distal portion that is coupled to and/or integrated with a frame of the nose cone 295, which may be disposed within/under a cover 299. In some implementations, the outer shaft 292 includes a plurality of cuts to provide flexibility for the shaft 292.

The handle 301 may include an outer housing 303 for manual gripping/engagement. The housing 303 of the handle 301 may be configured to be ergonomic, to be gripped by the user. The handle 301 may be configured to be manually translated to advance and retract the elongate shaft assembly 298 (e.g., comprising the outer sheath 391, outer shaft 292, and inner shaft 293). The handle 301 may be configured to be rotated about the longitudinal axis of the handle 301 and the shaft 298 to rotate and torque the elongate shaft 298. Such rotation may be desired to provide for a desired orientation of the implant to be deployed from the sheath 391. The handle 301 may include a release mechanism that is configurable to release the implant 32o from the delivery system 300.

The handle 301 may include a deflection mechanism configured to cause at least a portion of the elongate shaft 298 to deflect. The deflection may be in longitudinal planes extending outward from the longitudinal axis of the elongate shaft 298. Such deflection may be utilized to accommodate various bends in the patient's anatomy that may need to be navigated to deliver the implant to the desired location. The deflection mechanism may provide for a controllable deflection of the elongate shaft 298, as opposed to a passive deflection that may occur by simply passing a flexible shaft through bends in the patient's anatomy.

The outer shaft 292 may be proximally pulled/actuated in order to collapse the nose cone 275. For example, the outer shaft 292 may be welded or otherwise fastened to the nose cone structure 275, such as at a proximal collar or portion thereof, as described in detail above. The outer shaft/structure 292 may be actuated using a lead screw mechanism 305 associated with the handle 301. Alternatively, the outer shaft 292 may be manually pulled without the assistance of a lead screw or other actuator component.

The lead screw mechanism 305 may comprise a screw shaft 306 having threads configured to engage/mate with corresponding threads of a carrier/carriage component 307, the carrier component 307 being mechanically fixed to a proximal portion of the outer shaft 292. In some implementations, the outer shaft 292 is an integrated/unitary form or component with the carrier 307. By rotating the lead screw 306 through clockwise or counterclockwise rotation of a knob or similar actuator feature 302, the carrier 307 may be translated in a linear manner, such as in parallel with an axis of the lead screw 306, thereby causing linear actuation of the outer shaft 292. Linear actuation of the outer shaft 292 and results in a modification of the distance between the proximal end 294 of the nose cone frame 295 and the distal end 291 of the nose cone frame 295, which is mechanically coupled and/or fixed to the inner shaft 293.

Winding of the lead screw 306 may be translated to linear movement of the carrier 307, which is physically attached/coupled to the outer shaft 292. The knob 302 of the lead screw mechanism 306 can be accessible on any portion or area of the handle 301 or other structure. That is, although shown on a distal end portion of the handle, it should be understood that the actuator knob 302 may be positioned on a proximal end of the handle or in any other position relative to the handle 301. The outer shaft/member 292 may be driven back by the lead screw through rotation in one direction, whereas rotation in the opposite direction may cause advancement of the outer shaft 292, resulting in expansion of the nose cone 295.

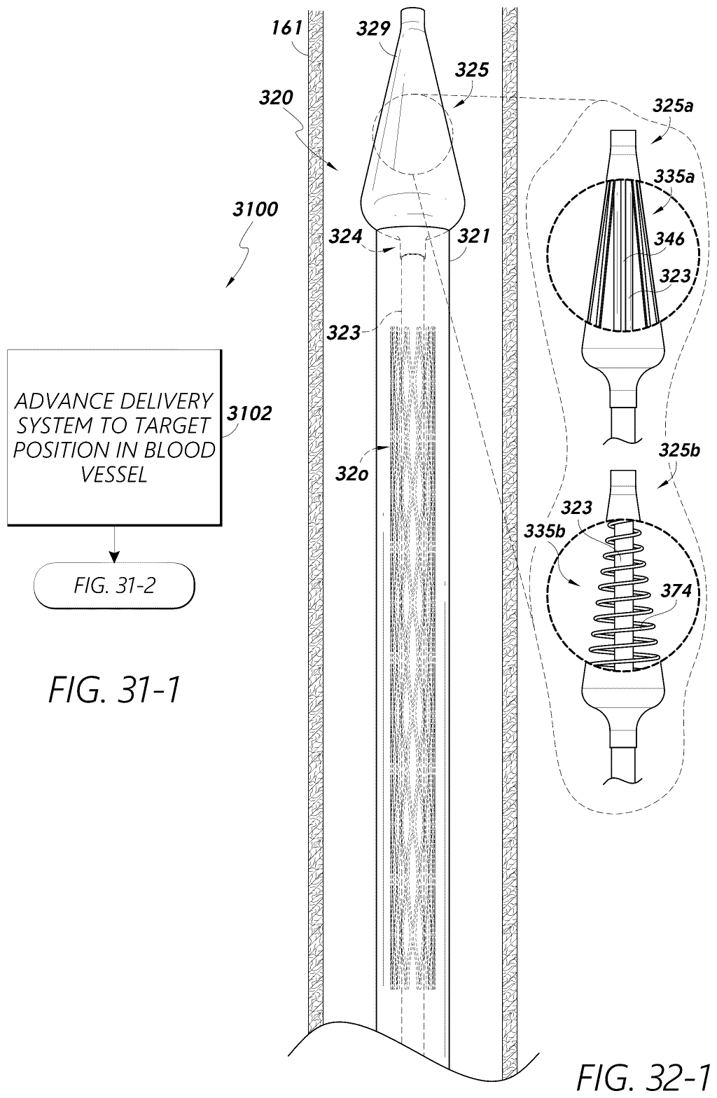

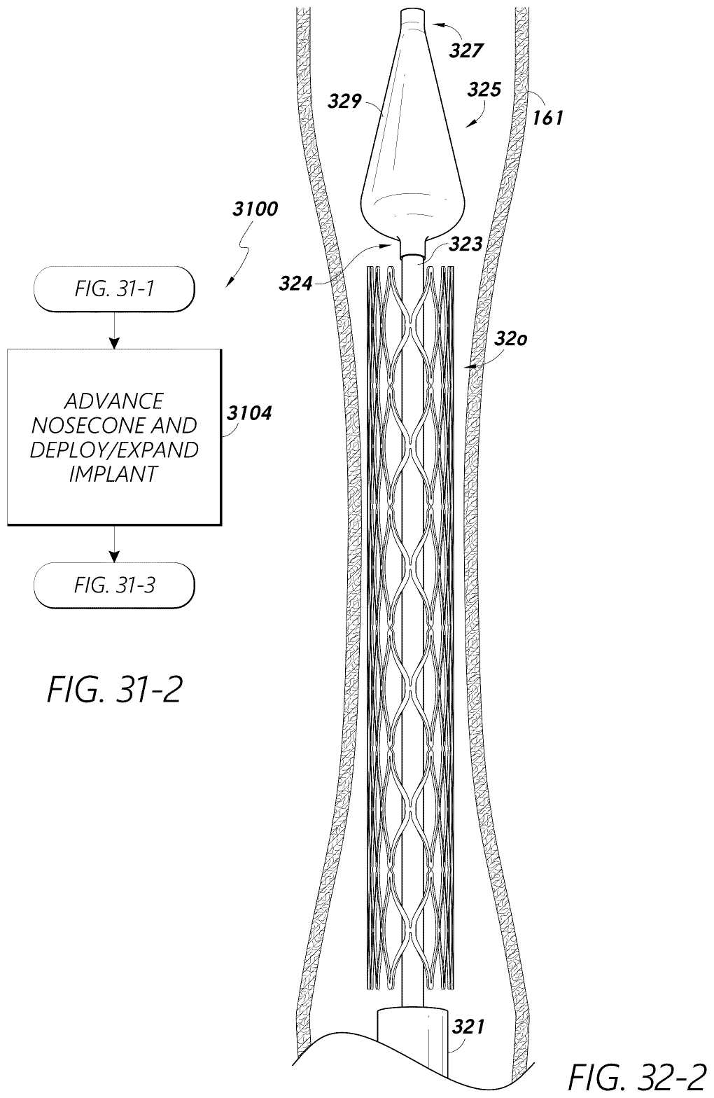

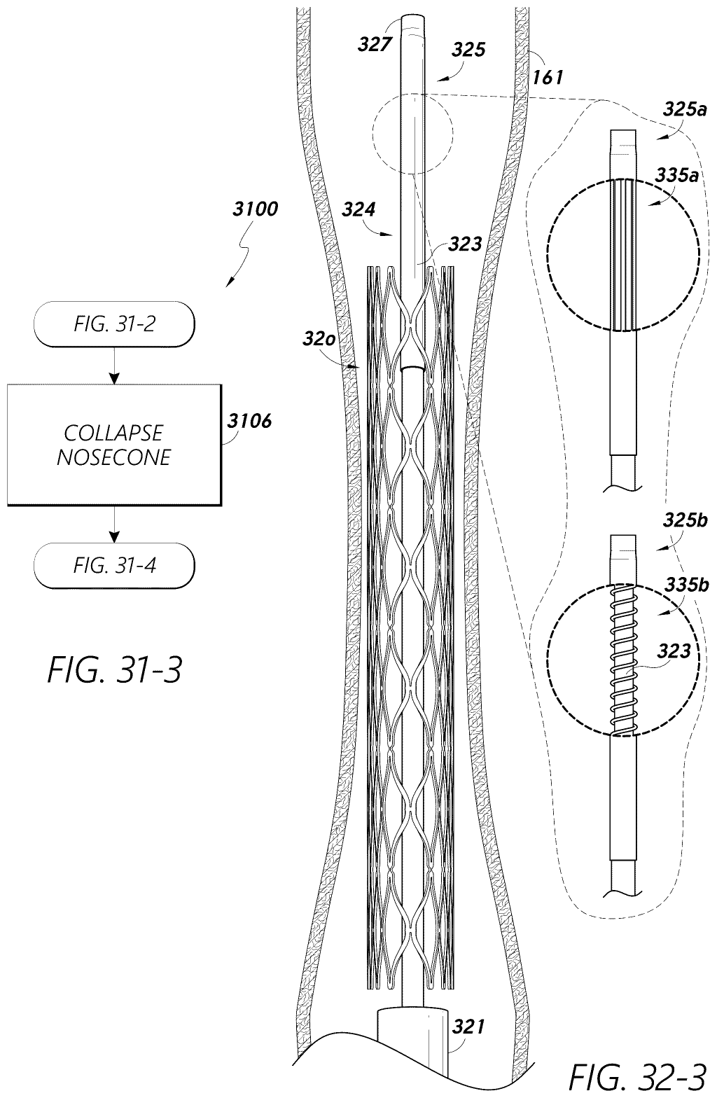

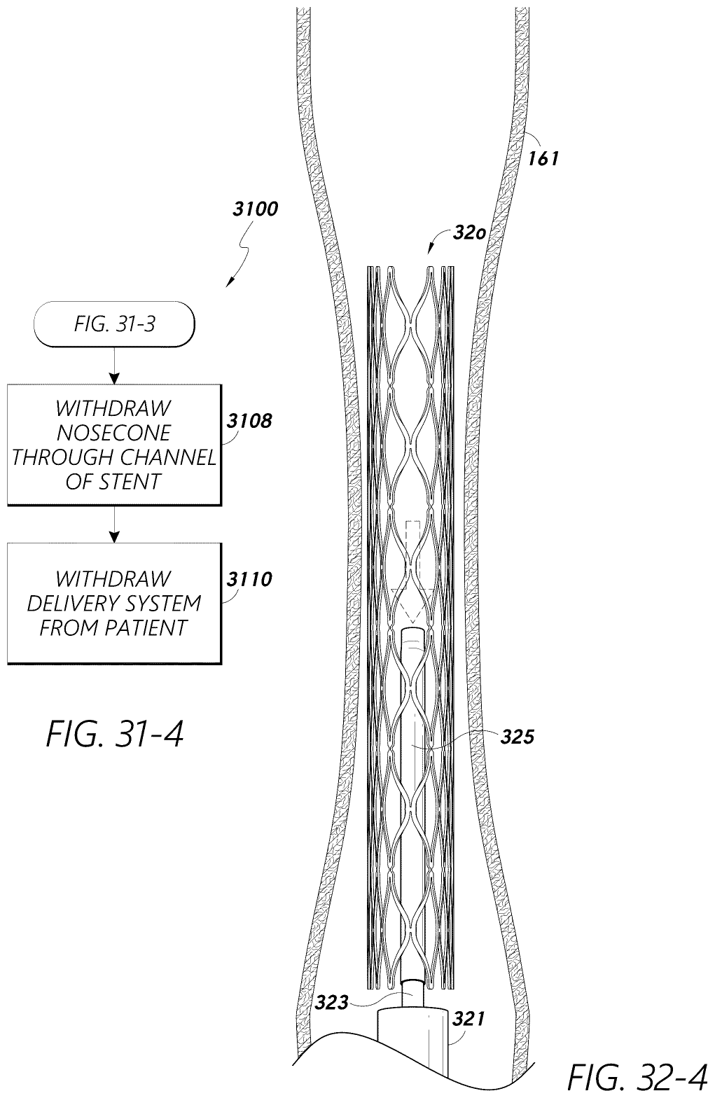

FIGS. 31-1, 31-2, 31-3, and 31-4 illustrate a flow diagram for a process 3100 for deploying an implant device 32o using a delivery system 32o with a collapsible nose cone 325 in accordance with one or more examples. FIGS. 32-1, 32-2, 32-3, and 32-4 provide images of the delivery system 32o and certain anatomy corresponding to operations of the process 3100 of FIGS. 31-1, 31-2, 31-3, and 31-4 according to one or more examples.

At block 3102, the process 3100 involves advancing the delivery system 32o to a target position in a blood vessel 161. For example, the delivery system 32o may comprise a distal nose cone feature 325 in accordance with any of the examples disclosed herein, wherein the nose cone 325 may lead the delivery system 32o to the target position in the patient anatomy. FIG. 32-1 shows the delivery system 32o disposed at least partially within the target blood vessel 161, wherein a stent 32o or other implant device is disposed in a distal portion/capsule of the delivery system 320. The delivery system 32o includes the collapsible nose cone 325 having a fluid-tight cover 329, such as a polymer cover or the like. The nose cone 325 may comprise a frame 335 configured to assume an expanded nose cone configuration and a collapsed/compressed configuration, as described above. For example, the nose cone frame 335 may comprise longitudinally oriented struts 346 and/or helically-wound coils 374, as described in detail above.

At block 3104, the process 3100 involves advancing the nose cone 325 and deploying/expanding the implant device 32o delivered using the delivery system 320. For example, the implant device 32o may be disposed on a nose cone shaft 323 extending proximally from the nose cone 325. FIG. 32-2 shows the deployed/expanded implant device 32o (e.g., oval/non-circular stent) with the nose cone 325 positioned distal of the implant 32o, whereas the distal end of the delivery system sheath/capsule 321 is positioned proximal of the deployed implant 32o. The nose cone shaft 323 runs through the implant device 32o between the nose cone 325 and the delivery system capsule/sheath 321.

At block 3106, the process 3100 involves collapsing the nose cone frame 335 to thereby reduce a profile of the nose cone 325. For example, collapsing the nose cone frame 335 may comprise pulling proximally on a proximal portion of the nose cone frame/wire 335 (and or distally pushing/advancing a distal portion/collar of the nose cone frame 335) in a manner as to cause mechanical radially-inward deflection of the struts/coils of the frame 335. Proximally pulling the nose cone frame 335 relative to the inner shaft/lumen of the delivery system 32o about which the frame 335 is disposed can be implemented by pulling a shaft or other structure mechanically coupled, interfaced, and/or integrated with/to a proximal portion/collar of the nose cone frame 335 relative to a shaft or other structure fixed to the distal portion of the frame 335. The inner shaft 323 (e.g., guidewire lumen) and/or outer shaft (e.g., tube or other structure coupled to the proximal end of the nose cone frame; not illustrated for visual clarity) may pass through a lumen or other portion of the shaft/adapter 323 over which the implant device is disposed when delivered to the target anatomy. In some implementations, collapsing the nose cone 325 may comprise actuating a lead screw actuator of a handle of the delivery system 320, or other actuator configured to effect a linear translation of the proximal portion 324 of the nose cone/frame 325 relative to the distal end 327 of the nose cone/frame. FIG. 32-3 shows the nose cone 325 in the collapsed, low-profile configuration.

At block 3108, the process 3100 involves withdrawing the nose cone 325 proximally through a channel/lumen of the implant device 32o and into and/or towards the distal portion/capsule of the delivery system sheath 321. FIG. 32-4 shows the collapsed nose cone 325 being pulled back through the stent/implant 325 lumen and towards/into the sheath 321 to retrieve the nose cone 325 and permit removal of the delivery system 320. At block 3110, the process 3100 involves withdrawing the delivery system 320 from the patient, thereby leaving the implant 32o in-place for therapeutic purposes.

### Additional Description of Examples

Provided below is a list of examples, each of which may include aspects of any of the other examples disclosed herein. Furthermore, aspects of any example described above may be implemented in any of the numbered examples provided below.

Example 1: An implant delivery system comprising an elongate sheath having an oval cross-sectional shape, and a tapered nose cone configured to engage a distal end of the elongate shaft, the nose cone having a base portion with an oval shape.

Example 2: The implant delivery system of any example herein, in particular example 1, wherein the nose cone is coupled to a nose cone shaft.

Example 3: The implant delivery system of any example herein, in particular example 1 or example 2, wherein the nose cone includes a first tapered portion having a first major-axis taper angle, and a second tapered portion having a second major-axis taper angle.

Example 4: The implant delivery system of any example herein, in particular example 3, wherein the first tapered portion is axially offset from the second tapered portion.

Example 5: The implant delivery system of any example herein, in particular example 3 or example 4, wherein the first tapered portion is associated with a distal tip of the nose cone, and the second tapered portion is associated a proximal base of the nose cone.

Example 6: The implant delivery system of any of any example herein, in particular any of examples 3-5, wherein the first tapered portion has a constant taper angle around a circumference of the first tapered portion.

Example 7: The implant delivery system of any example herein, in particular example 6, wherein the second tapered portion has a minor-axis taper angle that is less than the major-axis taper angle of the second tapered portion.

Example 8: The implant delivery system of any example herein, in particular example 7, wherein the minor-axis taper angle is less than 10°.

Example 9: The implant delivery system of any of any example herein, in particular any of examples 3-8, wherein the second tapered portion has minor-axis sidewalls that are parallel with an axis of the nose cone.

Example 10: The implant delivery system of any of any example herein, in particular any of examples 3-9, wherein the first tapered portion has a circular base, and the second tapered portion has an oval base.

Example 11: The implant delivery system of any of any example herein, in particular any of examples 1-10, wherein the nose cone comprises major-axis sidewalls that have a first taper angle relative to an axis of the nose cone, and minor-axis sidewalls that have a second taper angle that is less than the first taper angle.

Example 12: An implant delivery system comprising an elongate sheath, and a collapsible nose cone configured to be configured in a first configuration having a first maximum diameter and a second configuration having a second maximum diameter that is less than the first maximum diameter.

Example 13: The implant delivery system of any example herein, in particular example 12, wherein the nose cone is inflatable.

Example 14: The implant delivery system of any example herein, in particular example 13, further comprising a fluid lumen extending from the nose cone.

Example 15: The implant delivery system of any example herein, in particular example 14, wherein the fluid lumen extends a length of the elongate shaft to a reservoir external to the elongate shaft.

Example 16: The implant delivery system of any of any example herein, in particular any of examples 12-15, wherein the nose cone comprises a frame coupled at a first end portion thereof to an inner shaft, and a cover covering the frame.

Example 17: The implant delivery system of any example herein, in particular example 16, wherein the frame is shape-set in an expanded configuration.

Example 18: The implant delivery system of any example herein, in particular example 16 or example 17, wherein a second end portion of the frame is configured to slide over the inner shaft.

Example 19: The implant delivery system of any of any example herein, in particular any of examples 16-18, wherein the inner shaft comprises a guidewire lumen.

Example 20: The implant delivery system of any of any example herein, in particular any of examples 16-19, wherein the frame comprises a plurality of longitudinal struts.

Example 21: The implant delivery system of any example herein, in particular example 20, wherein the frame further comprises a distal collar coupled to distal ends of the plurality of longitudinal struts, and a proximal collar coupled to proximal ends of the plurality of longitudinal struts.

Example 22: The implant delivery system of any example herein, in particular example 20 or example 21, wherein proximal movement of the proximal collar relative to the inner shaft causes the plurality of longitudinal struts to deflect radially inward.

Example 23: The implant delivery system of any of any example herein, in particular any of examples 16-22, wherein the frame comprises a wire forming a helical coil.

Example 24: The implant delivery system of any example herein, in particular example 23, wherein the helical coil includes a plurality of winds of coil having increasing diameters moving proximally from a distal portion of the helical coil.

Example 25: The implant delivery system of any example herein, in particular example 23 or example 24, wherein pulling a proximal portion of the wire causes the helical coil to radially compress.

Example 26: The implant delivery system of any of any example herein, in particular any of examples 23-25, wherein the wire passes through a proximal portion of the cover.

Example 27: An implant delivery system comprising an elongate sheath having a first diameter, and a nose cone having a second diameter that is less than the first diameter, a flexible cover configured to cover a distal opening of the elongate sheath between a distal end of the elongate sheath and the nose cone, the cover having an opening configured to assume an expanded configuration and a contracted configuration.

Example 28: The implant delivery system of any example herein, in particular example 27, wherein the cover comprises a plurality of flaps that project distally from the distal end of the elongate sheath.

Example 29: The implant delivery system of any example herein, in particular example 27 or example 28, wherein the opening is formed by distal ends of the plurality of flaps.

Example 30: The implant delivery system of any of any example herein, in particular any of examples 27-29, wherein the cover is biased towards the contracted configuration of the opening.

Example 31: The implant delivery system of any of any example herein, in particular any of examples 27-30, wherein the cover is associated with the distal end of the elongate sheath.

Example 32: The implant delivery system of any example herein, in particular example 31, wherein the cover is disposed on an outer diameter of the distal end of the elongate sheath

Example 33: The implant delivery system of any example herein, in particular example 31 or example 32, wherein the cover is integrated with the distal end of the elongate sheath.

Example 34: The implant delivery system of any of any example herein, in particular any of examples 27-33, wherein the cover comprises a plurality of longitudinal slits.

Example 35: The implant delivery system of any of any example herein, in particular any of examples 27-34, wherein the cover comprises an elastic drape.

Example 36: The implant delivery system of any of any example herein, in particular any of examples 27-35, wherein the cover is associated with a proximal portion of the nose cone.

Example 37: The implant delivery system of any of any example herein, in particular any of examples 27-36, wherein the cover emanates proximally from the nose cone with the opening proximally-facing.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An implant delivery system comprising:
an elongate sheath having an inner diameter at a distal end thereof;
a nose cone shaft dimensioned for advancement within the elongate sheath; and
a collapsible nose cone associated with a distal end of the nose cone shaft and positionable at least partially beyond the distal end of the elongate sheath, the collapsible nose cone being transitional between an expanded state having a first maximum diameter and a compressed state having a second maximum diameter that is less than the inner diameter of the elongate sheath.

2. The implant delivery system of claim 1, wherein the nose cone is inflatable using a fluid lumen associated with the nose cone shaft.

3. The implant delivery system of claim 2, wherein the fluid lumen extends a length of the elongate sheath to a reservoir proximally external to the elongate sheath.

4. The implant delivery system of claim 1, wherein the nose cone comprises:
a nose cone frame coupled at a distal end portion thereof to an inner shaft portion of the nose cone shaft; and
a cover covering the nose cone frame.

5. The implant delivery system of claim 4, wherein a proximal end portion of the nose cone frame is configured to slide over the inner shaft portion.

6. The implant delivery system of claim 4, wherein the nose cone frame comprises a plurality of circumferentially spaced longitudinal struts configured to bend and radially deflect while maintaining a straight profile in a respective longitudinal axis plane.

7. The implant delivery system of claim 6, wherein the nose cone frame further comprises:
a distal collar coupled to distal ends of the plurality of longitudinal struts; and
a proximal collar coupled to proximal ends of the plurality of longitudinal struts.

8. The implant delivery system of claim 7, wherein proximal movement of the proximal collar relative to the inner shaft portion causes the plurality of longitudinal struts to deflect radially inward such that the plurality of longitudinal struts becomes flat and parallel with an axis of the inner shaft portion.

9. The implant delivery system of claim 5, wherein the nose cone frame comprises a wire forming a helical coil including a plurality of winds of coil having increasing diameters moving proximally from a distal portion of the helical coil.

10. The implant delivery system of claim 9, wherein pulling a proximal portion of the wire causes the helical coil to radially compress to form a tight, uniform-diameter coil around the inner shaft portion.

11. The implant delivery system of claim 1, wherein the nose cone comprises a flexible cover associated with a proximal portion of the nose cone, wherein the flexible cover is configured to:
assume an expanded configuration having the first maximum diameter in which the flexible cover extends from the proximal portion of the nose cone to cover a distal opening of the elongate sheath; and
radially contract toward an axis of the nose cone and around the nose cone shaft, resulting in a reduced profile for the nose cone.

12. An implant delivery system comprising:
an elongate sheath having a first diameter;
a nose cone shaft dimensioned for advancement within the elongate sheath;
a nose cone associated with a distal end of the nose cone shaft, the nose cone having a maximum diameter that is less than the first diameter; and
a flexible cover configured to cover a distal opening of the elongate sheath between a distal end of the elongate sheath and a proximal portion of the nose cone, the cover having an opening transitionable between an expanded configuration covering at least one of the distal end of the elongate sheath or a proximal portion of the nose cone and a contracted configuration against the nose cone shaft, the cover being biased to the contracted configuration.

13. The implant delivery system of claim 12, wherein the cover comprises a plurality of flaps that project distally from the distal end of the elongate sheath.

14. The implant delivery system of claim 13, wherein the opening is formed by distal ends of the plurality of flaps.

15. The implant delivery system of claim 12, wherein the cover comprises an elastic membrane.

16. The implant delivery system of claim 12, wherein the cover is integrated with the distal end of the elongate sheath in a unitary form.

17. The implant delivery system of claim 12, wherein the cover comprises a plurality of longitudinal slits.

18. The implant delivery system of claim 12, wherein:
the cover is associated with a proximal portion of the nose cone; and
the cover emanates proximally from the proximal portion of the nose cone with the opening facing proximally.

19. An implant delivery system comprising:
an elongate sheath having an oval cross-sectional shape; and
a tapered nose cone configured to engage a distal end of the elongate sheath, the nose cone having a base portion with an oval shape that conforms to the oval cross-sectional shape of the elongate sheath.

20. The implant delivery system of claim 19, wherein the nose cone includes:
a first tapered portion having a first major-axis taper angle; and
a second tapered portion having a second major-axis taper angle.
